(19) United States
(12) Patent Application Publication
(10) Pub. No.: US 2025/0195469 A1
(43) Pub. Date: Jun. 19, 2025

(54) COMPANION DIAGNOSTICS AND STRATEGIES FOR TREATMENT WITH AN ARYL HYDROCARBON RECEPTOR AGONIST COMPOUND

(71) Applicant: AZORA THERAPEUTICS, INC., Encino, CA (US)

(72) Inventors: Matthew DAVIDSON, Encino, CA (US); Julie SAIKI, Encino, CA (US)

(21) Appl. No.: 18/847,647

(22) PCT Filed: Mar. 16, 2023
(86) PCT No.: PCT/US2023/064599

§ 371 Date: Sep. 16, 2024

Related U.S. Application Data
(60) Provisional application No. 63/320,900, filed on Mar. 17, 2022.

(51) Int. Cl. A61K 31/404; A61K 31/05; A61K 31/405; A61K 31/407; A61K 31/427; A61K 31/4439; A61K 31/4704; C12Q 1/6883; C12Q 1/689; G01N 33/573; G01N 33/68

(52) U.S. Cl. CPC A61K 31/404; A61K 31/05; A61K 31/405; A61K 31/407; A61K 31/427; A61K 31/4439; A61K 31/4704; C12Q 1/6883; C12Q 1/689; G01N 33/573; G01N 33/6869; G01N 33/6875

(57) ABSTRACT

Methods for improving a therapeutic response and/or preventing adverse events that may occur upon or during treatment with a compound with activity as an aryl hydrocarbon receptor (AhR) agonist are described. An exemplary method comprises, prior to treating a subject with an AhR agonist compound, testing, or instructing to test, a sample from a subject to be treated for a diagnostic marker related to aryl hydrocarbon receptor biology. Based on a result from the testing one or more of (i) administering the AhR agonist compound, (ii) not administering the AhR agonist compound, and (iii) altering a dose or a dosing protocol of the AhR agonist compound is/are performed.

### CROSS-REFERENCE TO RELATED APPLICATIONS

This application claims the benefit of U.S. Provisional Application No. 63/320,900, filed Mar. 17, 2022, incorporated herein by reference in its entirety.

### TECHNICAL FIELD

The subject matter described herein relates to methods for improving clinical response rates and for preventing or mitigating adverse events that may occur due to treatment with an aryl hydrocarbon receptor (AhR) agonist.

### BACKGROUND

Aryl hydrocarbon receptor (AhR) agonists are a diverse set of both naturally occurring and synthetic small molecules that can bind to and activate the AhR. Activation results in a conformational change exposing a nuclear localization signal enabling translation to the nucleus and binding with the aryl hydrocarbon nuclear translocator (ARNT), also known as hypoxia-inducible factor-1β (HIF-1β), forming a heterodimer capable of modulating the transcription of many genes that express a consensus deoxyribonucleic acid (DNA) sequence known as a xenobiotic response element (XRE). Binding to XREs can modulate the expression of many genes that impact inflammation, development and metabolism.

Many of the signaling proteins that interact with the AhR pathway are shared with other signaling pathways. This is reflected by both canonical AhR signaling requiring ARNT as described above as well as non-canonical signaling that can occur in the absence of ARNT where agonized AhR can interact with the alternative proteins leading to alternative signals in response to AhR agonists. Signaling proteins involved in the AhR pathway are also utilized in other pathways, highlighted by how ARNT, which is also known as hypoxia induced factor-1-beta (HIF-1β), is required for HIF signaling, allowing survival in times of oxygen stress. These complex interactions may lead to unexpected adverse events (AEs) under some conditions of AhR signaling.

Once activated by an AhR agonist, some XRE responsive genes control aspects of metabolism via the expression of cytochrome P450 (CYP) enzymes, such as CYP1A1, CYP1A2, CYP1B1 and CYP19A1 or other enzymes including UDP-glucuronosyltransferase 1A6 (UGT1A6), and glutathione S-transferase A1 (GSTA1). Some of these same CYP enzymes are involved in the formation of and metabolism of estradiol (E2). In particular CYP1A1, CYP1A2 and CYP1B1 are involved in the generation of various hydroxyestradiols including 6α, 6β, 7α, 12β, 15α, 15β, 16α and 16β hydroxyestradiol from estradiol (E2) making these metabolites potential biomarkers of AhR activity.

Some of these CYPs can metabolize AhR agonists, creating a regulatory feedback loop. AhR agonists turn on AhR signaling that increases production of CYPs. This process can then metabolize the AhR agonists into non-bioactive metabolites. Other regulation on the AhR itself is triggered by production of inhibitory proteins including the aryl hydrocarbon receptor repressor (AhRR). Although activated AhR can be detected by nuclear colocalization of the AhR itself, this is challenging to do in a clinical setting as it requires both a biopsy and the ability to do immunohistochemistry to look for nuclear colocalization or the ability to determine the amount of AhR protein in the cytosolic vs nuclear fraction. Even when this can be done, it gives a relatively weak signal as AhR is protein is constantly shuttling back and forth between the cytosol and nucleus.

Thus, other more sensitive means of detecting AhR signaling can be used. CYP1A1 is routinely used as a surrogate for AhR signaling because it is highly conserved across species, has very low levels of basal expression, and can be rapidly and dramatically induced by AhR agonists.

Naturally occurring AhR agonists come from many sources including from dietary (ingestion of cruciferous vegetables high in indoles), environmental (ultraviolet light mediated production of tryptophan dimers), metabolic (kynurenine metabolites) or microbial sources (tryptophan-derived catabolites). Many of these AhR agonists derived from tryptophan can be found in the skin and gut. For example, the bacterial catabolism of tryptophan into molecules such as indol-3-acetic acid (IAA) in the gut. Reports have demonstrated a link between dysregulated tryptophan metabolism and autoinflammatory diseases such as psoriasis (Harden et al., Journal of Allergy and Clinical Immunology 137 (6): 1830-1840 (2016)), hidradenitis suppurativa (HS) (Guenin-Mace et al., JCI Insight 5(20):1-14 (2020)) and ulcerative colitis (Lamas et al., Nature Medicine 22 (6):598-605 (2016)); Sofia et al., Inflammatory Bowel Diseases 24 (7):1471-1480 (2018)) suggesting that dysregulated tryptophan metabolism and AhR signaling is associated with autoinflammatory disease.

AhR agonists have been used in the clinic to treat autoinflammatory diseases such as multiple sclerosis, ulcerative colitis, Crohn's disease, uveitis, psoriasis, nail psoriasis, and atopic dermatitis. However, not every patient responds to an AhR therapy, and response is difficult to predict for any given patient.

AhR agonists have been, and continue to be, explored in a pharmaceutical setting. There are a number of approved drugs with low levels of AhR activity including itraconazole, ketoconazole, omeprazole, 4-hydroxytamoxifen, raloxifene, tranilast, flutamide and leflunomide. Other higher potency AhR agonists, such as tapinarof, linomide (roquinimex), indirubin, 6-formylindolo[3,2−b]carbazole (FICZ) derivatives, and laquinimod, have been or are currently in development. Additional AhR agonists such as semaxanib, urolithin A, 2-(1′ H-indole-3′-carbonyl)-thiazole-4-carboxylic acid methyl ester (ITE), AQ-312, JNJ-2482272, DMI-9523 may be in development. Still others, such synthetic and natural products that contain indigo and indigo derivatives including but not limited to isoindigo, meisoindigo, and natura-alpha or prodrugs thereof have been explored as therapeutics. Derivates of any of the above, or related compounds, may be explored as therapeutics.

In a clinical setting, not every patient responds to AhR therapy and the magnitude of response and safety profile is often suboptimal. In phase 3 studies of tapinarof for the treatment of plaque psoriasis, only 35.2% to 40.2% had a meaningful clinical response as measured by the Physician's Global Assessment. Over 20% of patients developed inflammation of the hair follicle (folliculitis) (Lebwohl et al., New England Journal of Medicine 385(24):2219-2229 (2021)).

Further, prolonged use or exposure to AhR agonists in various clinical and non-clinical studies has resulted in reports of pulmonary arterial hypertension, folliculitis, pneumonitis, respiratory failure, myocardial ischemia, myocardial infarction, pericarditis, pleuritis, peritonitis, pneumonitis, chest pain, abdominal pain, diarrhea, vomiting, intussusception, joint pain, chest pain, back pain, liver damage, changes in liver enzymes, infection, peripheral neuropathy, headache, pancytopenia or leukopenia.

Clinically, pulmonary arterial hypertension has been reported in published case reports after prolonged use of indigo naturalis, a botanically-derived traditional medicine which contains a number of AhR agonists.

Pulmonary arterial hypertension (PAH) is a progressive disease characterized by increased pulmonary vascular resistance, leading to chronic elevation in pulmonary arterial pressure resulting from restricted flow through the pulmonary arterial circulation. These pathobiological features may lead to right-sided heart failure and premature death. PAH may be idiopathic, heritable, and associated with many conditions (connective tissue disease, human immunodeficiency virus or congenital heart disease). In some cases PAH is associated with drugs or toxins. Drugs which induce the AhR including indigo naturalis have been associated with the development of PAH. In histological section of lungs from patients with PAH CYP1B1 and CYP19A1 were highly expressed suggesting a potential link between PAH, CYP expression and lung exposure to AhR agonists (White 2012).

Other serious side effects have occurred in late-stage trials of long-acting, systemic AhR agonists for the treatment of multiple sclerosis. In Phase 3 trials of laquinimod, there were seven reported cases of non-fatal, myocardial ischemia in the high dose (1.2 mg/day) group but not the low dose group (0.6 mg/day) in the treatment of relapsing-remitting multiple sclerosis. Ultimately the high dose of laquinimod was dropped and the low dose failed to meet the trial's primary endpoint. Linomide (roquinimex) was in Phase 3 trials, but development was terminated due to cardiovascular toxicity, specifically pleuritis, pericarditis, and chest pain.

Although there is substantial promise for the therapeutic use of AhR agonists, the pharmacological causes leading to the development of these reported side effects and methods to mitigate or prevent them are lacking. Methods for enhancing the response rate, and for preventing and mitigating adverse effects from administration of AhR agonists are needed.

### BRIEF SUMMARY

The following aspects and embodiments thereof described and illustrated below are meant to be exemplary and illustrative, not limiting in scope.

In one aspect, a method for increasing response and/or response rate to treatment with an aryl hydrocarbon receptor (AhR) agonist is provided. In another aspect, a method for preventing or mitigating an adverse event prior to or during treatment with a compound with activity as an aryl hydrocarbon receptor (AhR) agonist is provided. In another aspect, a method for preventing or mitigating an adverse event prior to treatment with a compound with activity as an aryl hydrocarbon receptor (AhR) agonist is provided.

In an aspect, a method for treatment with an aryl hydrocarbon receptor (AhR) agonist compound comprises, prior to treating a subject with an AhR agonist compound, providing or instructing to provide a sample from a subject and testing or instructing to test the sample for a diagnostic marker related to aryl hydrocarbon receptor biology. Based on a result from the testing or the instructing to test, performing one or more of (i) administering the AhR agonist compound, (ii) not administering the AhR agonist compound, and (iii) altering a dose or a dosing protocol of the AhR agonist compound.

In one embodiment, the testing or instructing to test is for a diagnostic marker selected from the group consisting of:
a. an AhR agonist compound in a sample of skin, stool, urine, or plasma obtained from the subject;
b. tryptophan in a sample of skin, stool, urine or plasma obtained from the subject;
c. a cytochrome P450 (CYP) enzyme selected from CYP1A1, CYP1A2, CYP1B1, and CYP19A1 in a colon sample or biopsy, an intestinal biopsy, a skin biopsy or peripheral blood mononuclear cells (PBMCs) obtained from the subject;
d. an aryl hydrocarbon receptor repressor (AhRR) mRNA or protein in a colon sample or biopsy, an intestinal biopsy, a skin biopsy or peripheral blood mononuclear cells (PBMCs) obtained from the subject;
e. nuclear localizing AhR in a colon sample or biopsy, an intestinal biopsy or a skin biopsy obtained from the subject;
f. a level of IL-22 mRNA or protein in a sample of plasma, a colon sample of biopsy, an intestinal biopsy or a skin biopsy obtained from the subject; and
g. a polymorphism in caspase recruitment domain 9 (CARD9) in a sample taken from the subject.

In an embodiment, the AhR agonist compound in the sample from the subject is a level of AhR agonist compound(s) in the sample with activity and/or potential to agonize AhR to give a total level of AhR agonist compound(s) in the sample, prior to treating with the AhR agonist compound. In an embodiment, the tryptophan in the sample from the subject is a level of tryptophan in the sample. In embodiments, the sample is from the subject prior to treatment with the AhR agonist compound. In embodiments, the diagnostic marker is tested and quantified from the sample obtained from the subject prior to treatment with the AhR agonist compound to give a baseline level of the diagnostic marker prior to treatment with the AhR agonist compound.

In one embodiment, the method achieves an improvement in response to treatment with the AhR agonist compound that is at least about 10%, 15%, 20%, 25%, 30%, 35%, 40%, 45%, 50%, 55%, 60%, 65%, 70% or 75% increased relative to a subject treated with the same AhR agonist compound that did not receive a test for the diagnostic marker.

In an aspect, a method for preventing or mitigating an adverse event prior to or during treatment with a compound with activity as an aryl hydrocarbon receptor (AhR) agonist comprises prior to or while treating a subject with an AhR agonist compound, providing or instructing to provide a sample from a subject and testing or instructing to test the sample for a diagnostic marker related to aryl hydrocarbon receptor biology. Based on a result from the testing or the instructing to test, one or more of the following is/are performed:
i. administering the AhR agonist compound according to a schedule selected to reduce likelihood or severity of an adverse event;
ii. administering the AhR agonist compound at a dose selected to reduce likelihood or severity of an adverse event;
iii. not administering the AhR agonist compound;
iv. administering a treatment other than an AhR agonist; and
V. administering an AhR antagonist or a compound that induces CYP.

In one embodiment, the testing or instructing to test is for a diagnostic marker selected from the group consisting of:
a. a polymorphism in AhR, ARNT, HIF-1α or HIF-2α, AhRR, BMPR2, CYP1A1, CYP1A2, CYP1B1 and CYP19A1 or a promoter thereof;
b. ability for microsomes or cells from the subject to metabolize an AhR agonist in vitro or in vivo; and
c. ability of an AhR agonist to induce AhR translocation to the nucleus or to induce CYP1A1, CYP1A2, CYP19A1 and/or CYP1B1 expression in vitro or in vivo.

In one embodiment, the performing comprises (i), (ii) or (v), and the administering according to (i), (ii) or (v) reduces occurrence or severity of an adverse event in a subject compared to subjects that are not administered the AhR agonist compound or a compound that induces a CYP enzyme.

In one embodiment, administering according to (i), (ii) or (v) reduces occurrence or severity of an adverse event in a subject by at least about 10%, 15%, 20%, 25%, 30%, 35%, 40%, 45%, 50%, 55%, 60%, 65%, 70% or 75%.

In an aspect, a method for preventing or mitigating an adverse event during treatment with a compound with activity as an aryl hydrocarbon receptor (AhR) agonist comprises during treatment of a subject with an AhR agonist compound, providing or instructing to provide a sample from a subject and testing or instructing to test the sample for a diagnostic marker related to aryl hydrocarbon receptor biology. Based on a result from the testing or the instructing to test, one or more of the following is/are performed:
i. administering the AhR agonist at a dosing frequency that is reduced relative to a clinically approved dosing frequency or a previously planned dosing frequency;
ii. administering the AhR agonist at a dose that is reduced relative to a clinically approved dose or a previously planned dose;
iii. administering an initial starting dose of the AhR agonist followed by a subsequent dose that is greater than the initial starting dose;
iv. discontinuing use of the AhR agonist;
v. continuing use of the AhR agonist in conjunction with safety monitoring;
vi. administering a treatment other than an AhR agonist;
vii. co-administration of an AhR antagonist or of a compound that induces CYPIA, CYP1A2, CYP1B1 or CYP19A1;
viii. co-administration of a concomitant medication;
ix. discontinuing of a concomitant medication, food, drug or activity; and
x. no change in treatment.

In one embodiment, the testing or instructing to test is for a diagnostic marker selected from the group consisting of:
a. plasma, urine, colon or skin biopsy level of the administered AhR agonist or a metabolite thereof;
b. a plasma liver enzyme, such as alanine aminotransferase (ALT) or aspartate aminotransferase (AST);
c. PBMC expression of nuclear AhR or increased AhRR, CYP1A1 CYP1A2, CYP1B1 or CYP19A1 and/or CYP1B1 expression relative to a healthy subject;
d. systolic blood pressure, mean pulmonary arterial pressure (mPAP), pulmonary arterial systolic pressure (PASP), left atrial dimension (LAD), left ventricle end-diastolic diameter (LVEDD), left ventricular ejection fraction (LVEF), inferior vena cava dimension, right atrial area (RA area), tricuspid annular plan systolic excursion (TAPSE), tricuspid regurgitation pressure gradient (TRPG), aortic regurgitation, mitral regurgitation, valve thickening, restrictive valve motion, shortness of breath and/or fatigue;
e. hematocrit or plasma erythropoietin (EPO) level; and
f. plasma or urine level of serotonin, melatonin, 17B-estradiol (E2) or 16α-hydroxyestrone.

In one embodiment, the performing comprises (i), (ii), (iii), (v), (vii), (viii), or (ix), and the administering according to (i), (ii) or (v), the continuing according to (v), the co-administration according to (vii) or (viii) and/or the discounting according to (ix) reduces occurrence or severity of an adverse event in a subject compared to subjects that are not administered the AhR agonist compound, not administered a compound that induces a CYP enzyme or a concomitant medication, and/or continuing a concomitant medication, food, drug or activity.

In one embodiment, the method reduces occurrence or severity of an adverse event in a subject is by at least about 10%, 15%, 20%, 25%, 30%, 35%, 40%, 45%, 50%, 55%, 60%, 65%, 70% or 75%.

In one embodiment, continuing use of the AhR agonist in conjunction with safety monitoring comprising continuing use with a safety monitoring program selected from an echocardiogram, a walk test for a defined period of time and a questionnaire regarding shortness of breath and/or ability to exercise without fatigue.

In an aspect, a method for preventing or mitigating an adverse event prior to or during treatment with a compound with activity as an aryl hydrocarbon receptor (AhR) agonist is provided. The method comprises consulting a product label that prohibits certain concomitant medications, drugs, foods, diets, supplements and/or activities that may increase AhR signaling, increase AhR agonist absorption or interfere with the ability to metabolize an AhR agonist, and based on the consulting, determining whether to administer, to not administer or to administer with an adjusted a dose range or frequency of the AhR agonist to a subject based on likelihood of an adverse event for the subject.

In an aspect, a method for preventing or mitigating an adverse event prior to or during treatment with a compound with activity as an aryl hydrocarbon receptor (AhR) agonist comprises consulting a product label that contraindicates the use of the AhR agonist in patients with known medical conditions, predispositions or family history that may increase the likelihood of developing a cardiovascular disease, including pulmonary arterial hypertension and valvular heart disease.

In an aspect, a method for reducing systemic exposure or Cₘₐₓ of an AhR agonist comprises administering a compound that induces a CYP enzyme capable of metabolizing a to-be-administered AhR agonist, wherein the administering is prior to administration of an oral dosage form containing an AhR agonist.

In one embodiment, the compound that induces a CYP is an AhR agonist. In one embodiment, the AhR agonist that induces a CYP is the same as the AhR agonist in the oral dosage form, wherein the AhR agonist that induces a CYP is administered at a dose lower than a dose of the AhR agonist in the oral dosage form. In one embodiment, the administering reduces occurrence or severity of an adverse event in a subject compared to subjects that are not administered a compound that induces a CYP enzyme. In one embodiment, the administering reduces occurrence or severity of an adverse event in a subject by at least about 10%, 15%, 20%, 25%, 30%, 35%, 40%, 45%, 50%, 55%, 60%, 65%, 70% or 75%.

In one embodiment, the adverse event for any of the methods described herein is selected from the group consisting of folliculitis, essential hypertension, pulmonary hypertension, pulmonary arterial hypertension, pneumonitis, respiratory failure, myocardial ischemia, myocardial infarction, pericarditis, pleuritis, peritonitis, pneumonitis, chest pain, abdominal pain, diarrhea, vomiting, intussusception, joint pain, back pain, liver damage, changes in liver enzymes, infection, peripheral neuropathy, headache, pancytopenia, and leukopenia.

In an aspect, a method of treating one or more patients with an AhR agonist comprises providing a data storage facility comprising a database of patient records, each patient record having a medication authorization field for entering a first prescription for the AhR agonist to treat the patient; a central controller having one or more processors coupled to a communication network, which central controller is coupled to the data storage facility to read and write data to the data storage facility via the network. The central controller controls transmission and receipt of data to and from the data storage facility via the network, the central controller is programed to output via the network a first authorization of a first prescription of AhR agonist to a patient previously subjected to one or more initial medical tests, each providing an initial medical test result. The initial medical test is selected from the group consisting of a medical examination by a physician, a genetic test, a physiological function test, and a medical imaging test. Output of the first authorization is dependent upon satisfactory results of one or more of the initial medical tests entered into each patient's record, and further programed to schedule one or more subsequent tests for each patient prior to allowing entry of a prescription in the medication authorization field. At least one of the subsequent medical tests is an echocardiographic imaging test which echocardiographic imaging test is performed in a manner which provides measurements of dimensions of one or more internal heart structures and heart flow-rate, and the patient receives or continues to receive medication only on entry of satisfactory echocardiography assessment results. The central controller inhibits the authorization output of the first or subsequent prescriptions upon the entry of unsatisfactory test results. The central controller manages one or more aspects of the authorized prescription for the patient selected from the group consisting of dosage amount; dosing regimen; and intended time period of use, whereby overuse or misuse of the AhR agonist is inhibited and wherein aggregated and analyzed data is reported to a regulatory agency.

In certain embodiments, a diagnostic value(s) used in the methods is determined by comparing to a reference value, standard or sample. In other embodiments, the diagnostic value is determined by an amount changed or different from a reference value, standard or sample, where the amount changed is a decrease or increase in the amount of diagnostic value relative to the reference. In one embodiment, the change is a statistically significant change. In one embodiment, the change is a statistically significant reduction in the diagnostic value relative to a reference (the reference can be, for example, a baseline value, standard, historical control, disease matched cohort or healthy control cohort).

In other embodiments, the diagnostic value is a biomarker or plasma concentration of a compound.

In some cases, the diagnostic value or biomarker is present in an amount that is about 10%, about 15%, about 20%, about 25% or about 30% different than the amount of a reference sample, value or standard. In one embodiment, the amount in the sample is a decreased amount relative to the reference. In another embodiment, the different amount in the sample is an increased amount relative to the reference. For example, a biomarker that is a polymorphism, the marker can be determined to be present, absent or heterozygous. A marker that is a metabolite may be evaluated with respect to a change from a standard group or reference sample.

In embodiments, the reference sample, standard or value is a standard laboratory value or range of values for the marker in a human. In another embodiment, the reference sample, standard or value is a reference genome. In another embodiment, the reference sample, standard or value is a baseline concentration amount or range for the marker in a human. In other embodiments, the reference sample, standard or value is a tissue or fluid sample from the human subject or patient prior to treatment with an AhR agonist compound (a baseline). In other embodiments the reference sample, standard or value is a tissue or fluid sample from the human subject or patient during the course of treatment with an AhR agonist compound. In other embodiments, the reference sample, standard or value is a collection or population of biological samples from healthy subjects or similarly disease-matched subjects, each sample evaluated for the marker where the average value for the marker in the collection or population is determined and is taken as the reference sample, standard or value.

In one embodiment, a diagnostic test comprises detecting in a sample a concentration of the administered AhR agonist compound or a metabolite thereof.

In the above embodiment, a diagnostic test is performed on a blood, serum or plasma sample from the subject.

In another embodiment, the diagnostic testing comprises evaluating DNA in the sample for a polymorphism in the AhR, AhRR, CYP1A1, CYP1A2, CYP1B1 or their respective promoters or other genes or gene products.

In yet another embodiment, the testing comprises evaluating (i) DNA in the sample for a polymorphism in a CYP450 enzyme, (ii) a xenobiotic metabolic enzyme regulated by a xenobiotic response element, (iii) a polymorphism in a xenobiotic response element that modulates AhR function, and/or (iv) a polymorphism in the gene BMPR2.

In an embodiment, the xenobiotic metabolic enzyme is CYP1A1, CYP1A2 or CYP1B1.

In another embodiment, the testing evaluates more than one polymorphism to assign a polymorphism risk score.

In another embodiment, the testing comprises genotyping via whole genome sequencing, targeted sequencing, exome sequencing or detection via PCR for a specific polymorphism.

In still another embodiment, the testing comprises detecting in microsomes or cells from the sample an ability to metabolize an AhR agonist.

In an embodiment, the cells or microsomes are from a sample that is blood, skin, vasculature, liver, intestine, color or lung.

In yet another embodiment, the testing comprises detecting binding of an AhR agonist to a recombinant, polymorphic AhR.

In another embodiment, the testing comprises detecting mRNA expression or protein levels of CYP1A1, CYP1A2 or CYPIB1.

In another embodiment, the testing comprises detecting in microsomes or cells from the sample an ability of xenobiotic metabolizing enzymes to mediate deethylation of 7-ethoxyresorufin substrate to resorufin (the EROD assay).

In another embodiment, the testing comprises detecting in a blood or plasma sample a level of an enzyme indicative of AhR activation or liver toxicity.

In one embodiment, the enzyme is alanine aminotransferase or aspartate aminotransferase.

In another embodiment, the testing comprises detecting in the sample presence or absence of a gene product regulated by a xenobiotic response element.

In one embodiment, gene is selected from the group consisting of CYP1A1, CYP1A2, CYP1B1 IGFBP10, and GSTP1.

In an embodiment, the testing comprises polymerase chain reaction, quantitative polymerase chain reaction, loop mediated isothermal amplification (LAMP), genotyping including whole genome sequencing, targeted sequencing, exome sequencing, microarray, RNA-sequencing, mass spectrometry, immunohistochemistry, or immunofluorescence.

In still another embodiment, the testing comprises detecting in the sample presence or absence of a metabolic or catabolic product regulated by enzymes regulated by AhR. In an embodiment, the product is selected from the group consisting of serotonin, tryptophan, melatonin, 5-hydroxy-L-tryptophan (5-HTP), 5-hydroxyindoleacetuc acid (5-HIAA), 17β-estradiol (E2) or 16α-hydroxyestrone, a steroid, or a hormone,

In one embodiment, the diagnostic testing comprises analyzing or inspecting for a lack proteins or cells induced by activated hypoxia induced factor.

In an embodiment the protein is erythropoietin (EPO).

In another embodiment, the lack of cells are red blood cells and they are detected by analyzing or inspecting for the relative volume or concentration of red blood cells via hematocrit or complete blood cell count.

In some embodiments, the method further comprises administering to a subject sample a compound metabolized by an enzyme induced by an AhR agonist to generate a metabolite, and detecting the metabolite. In an embodiment, the compound is 7-ethoxyresorfin, the enzyme induced by the AhR agonist is CYP1A1 and the metabolite is resorfin.

In still other embodiments, a subject is one undergoing a lifestyle activity that potentially activates the aryl hydrocarbon receptor. The lifestyle activity, may be smoking, pollutant exposure, hypoxic or hyperoxic conditions, and/or ingestion of foods, supplements or drugs with AhR activating potential.

In other embodiments, the subject is one with a lifestyle activity that inhibits metabolism of an AhR agonist. The activity may inhibit the activity of a CYP enzyme. In some embodiments, the lifestyle activity, may be ingestion of bergamottin, grapefruit, inhalation of marijuana or a component thereof such as tetrahydrocannabinol or cannabidiol or consumption or use of concomitant medications, drugs or alcohol.

In other embodiments, the subject is one with a lifestyle activity that increases absorption of an AhR agonist. The lifestyle activity, may be a high fat or high alcohol diet.

In yet other embodiments, the subject is one at risk of reduced or altered metabolism of an AhR agonist compound.

In an embodiment, the genetic polymorphism in CYP1A1, CYP1A2, or CYPIB1 leads to reduced metabolism of the AhR agonist.

In other embodiments, the method further comprises treating or recommending to treat the subject with a drug that increases the metabolism or the clearance of the AhR agonist.

In the above embodiment, the drug which increases AhR metabolism or clearance may be given prior to beginning therapy with the AhR agonist without the explicit need for a diagnostic.

The drug which increases AhR metabolism or clearance may be a CYP inducer. The drug which increases AhR metabolism or clearance may itself be an AhR agonist. Alternatively, the drug which increases AhR metabolism or clearance may not AhR agonist. The drug which increases AhR metabolism or clearance may itself be the same AhR agonist as is to be dose therapeutically. The drug which increases AhR metabolism or clearance may be a lower dose or a less bioavailable form of the AhR agonist to be administered for therapeutic use. This lower dose or less bioavailable form of the AhR agonist may result in induction of a CYP locally at high levels without inducing high levels of systemic CYP expression. The local expression of CYPs may occur in the gastrointestinal tract and/or liver and not in the lungs.

This strategy of pre-administration of a compound prior to therapeutic dosing with an AhR agonist without the need of a diagnostic test may be referred to as “lead in dosing”, “run in dosing”, “priming dosing”, “starting dose”, “initial dosing”, “metabolic preactivation dosing” or even as a “reverse loading dosing”. Approved drugs utilizing this strategy in order to induce local metabolic activation in order to reduce subsequent systemic exposure are not heretofore known. Strategies wherein a dose is titrated up or down until it is not well tolerated and/or where a desired therapeutic outcome is observed are known. A lower initial dose intended for metabolic pre-activation of a subsequently administered drug makes sense for orally administered drugs that are intended for local activity in the gut that can be metabolized by local first-pass metabolism where systemic exposure is not desired, which encompasses a small and novel pharmacological space, hence why it has not been disclosed in the art previously.

The disclosure herein related to use of an oral AhR agonist and a lead in dose for use in the gastrointestinal tract, finds use additionally to limit systemic exposure with other forms of local administration such as to the eye, ear, skin, vagina, or lungs.

In other embodiments, the method further comprises treating or recommending to treat the subject with a drug that increases activity of CYP1A1, CYP1A2, or CYP1B1.

In other embodiments, an adverse event of interest is selected from the group consisting of folliculitis, essential hypertension, pulmonary arterial hypertension, pneumonitis, respiratory failure, myocardial ischemia, myocardial infarction, pericarditis, pleuritis, peritonitis, pneumonitis, chest pain, abdominal pain, diarrhea, vomiting, intussusception, joint pain, back pain, liver damage, changes in liver enzymes, infection, peripheral neuropathy, headache, pancytopenia, and leukopenia.

In other embodiments, an AhR agonist compound is selected from the group consisting of itraconazole, FICZ, ITE, urolithin A, semaxanib, ketoconazole, omeprazole, 4-hydroxytamoxifen, raloxifene, tranilast, flutamide, leflunomide, tapinarof and tapinarof derivatives, linomide (roquinimex), laquinimod, Aquilion AQ-312, DMI Bioscience DMI-9523, Resolvex RLV-102, 2-(1′H-indole-3′-carbonyl)-thiazole-4-carboxylic acid methyl ester (ITE), JNJ-2482272, FICZ derivatives like Nogra NPD-0614-02 and NPD-0614-24,Nexys NEX003, Artus ARTX-28, indigo naturalis, indigo, indirubin or any derivatives thereof. Specific examples of indigo derivatives include, but are not limited to, isoindigo, meisoindigo, and natura-alpha.

In some aspects, the present methods relate to use of a diagnostic test that relies on genetic, enzymatic and/or pharmacokinetic parameters related to an AhR agonist, the activation of the AhR and/or the activation or inhibition and/or increase or decrease of metabolic enzymes, transporters, downstream messengers, metabolites, hormones or enzymes.

Once a diagnostic value or diagnostic values has/have been obtained, in some embodiments, it or they can be used by the patient or clinician in a number of ways including, maintaining treatment, stopping treatment, altering treatment, co-administering treatment, changing safety monitoring, changing dose, changing dosing schedule, determination of a starting dose, or some combination of the above where appropriate.

In addition to the exemplary aspects and embodiments described above, further aspects and embodiments will become apparent by study of the following descriptions.

Additional embodiments of the present methods will be apparent from the following description and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The methods of the present disclosure can comprise, consist essentially of, or consist of, the components disclosed.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to describe the state of the art more fully as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The methods described herein may be used with any biologic sample including but not limited to skin, sebum, mucus, sperm, whole blood, plasma, white blood cells, peripheral blood mononuclear cells, red blood cells, colon, intestine, stool, urine, tears, liver, lung, endothelium, sputum, saliva, oral swab, and/or nasal swab.

The results from the diagnostics described herein may be used alone or in combination. The results of the diagnostics may be combined with other patient or historical control information in order to inform treatment decisions. These treatment decisions may be to administer, not administer, or alter the dose, duration or route of administration of an AhR agonist or AhR antagonist for the treatment of a human disease.

Throughout the disclosure reference to measuring the activity of various gene products is made. In the examples and claims one may substitute the gene product being measured as the mRNA, protein or enzymatic activity in any example. For example, CYP1Al may be measured at the mRNA level via qPCR, at the protein level via western blot or at the enzymatic level via the EROD assay. The specific method of measurement may be substituted with another means of measurement in any example.

Along the same lines, the examples given are not meant to be limiting. If one is measuring an AhR regulated gene activity in one tissue, one could substitute another AhR regulated gene in a different tissue. For example, CYP1A1, in the skin may be substituted with CYP1A2 in the colonic epithelium.

The magnitude of change in the examples is not meant to be rate limiting. The meaningful change will depend on the specific diagnostic assay. A meaningful change to inform a treatment decision may be a change from baseline, a change from matched controls, a change from a reference value from a healthy individual, a change from a non-involved area or some other change. The change may be more or less than 1%, 10%, 25% 50%, 75%, 90% or 99%. The change may be based on an average, a standard deviation, a cutoff, an interquartile range or some other meaningful change. The changes may be based on meaningful changes in a database, a clinical trial, previous experiments or intra-experimental controls. The changes may be based on high or low values. The changes may or may not be statistically significant.

The term coadministration may be concurrent or immediately before or after administration. In some instances it may mean within in 1, minute, 10 minutes, 1 hour or 1 day to administration of something else.

### II. Methods

Data in patients with autoimmune inflammatory diseases often show a correlation between levels of AhR agonist and/or AhR signaling and disease. In some cases, reduced AhR agonists in the plasma or tissue compared to a healthy patient as a control correlates with severity of disease. It is not known if low levels of AhR agonists and/or AhR signaling at baseline, prior to treatment with an AhR agonist, is predictive of a patient's clinical response to AhR agonists for the treatment of inflammatory diseases. If the level of AhR agonist or AhR signaling is correlated with a response, response rate, or magnitude of response to the administration of AhR agonists, a companion diagnostic measuring this would enable more efficient clinical trials, enhanced clinical response and response rates, and/or a greater magnitude of response to administration of AhR agonists. It could also be used to define a target patient population and minimize the number of non-responders, saving costs and medical resources and enable AhR-agonist non-responders to receive other interventions that may be of greater benefit.

The present methods, in some embodiments, relate to determining, analyzing and/or correlating a subject's response to AhR agonist therapy with the amount of baseline AhR signaling and/or expression level(s) of endogenous AhR agonist(s). The methods also relate to enhancing or improving a subject's response to treatment with an AhR agonist compound. In other embodiments, the present methods relate to stratifying patients based on their level of AhR signaling, the presence of certain or total AhR agonists, the activity or abundance of certain metabolic enzyme(s) induced by AhR signaling and/or the level(s) of AhR agonist precursor molecules. The methods have utility as a diagnostic that can enhance or improve the response or response rate to therapy with an AhR agonist to treat, for example, an inflammatory disease.

Some genetic mutations result in a decreased ability to produce or respond to AhR agonists. For example, inflammatory bowel disease (IBD) associated risk alleles in caspase recruitment domain 9 (CARD9) fail to appropriately metabolize tryptophan and related molecules leading to defects in production of AhR agonists. These patients may be more responsive to administration of AhR agonists than CARD9 normal IBD patients.

Some tryptophan-derived AhR agonists can be made by the microbiota. For example, certain species of bacteria and fungi are known to be capable of producing indigo and indirubin. Patients who lack or have low abundance of these microbes may be more responsive to administration of AhR agonists.

In response to AhR agonist administration, intestinal epithelial cells make the anti-inflammatory cytokine interleukin 22 (IL-22) (Lamas 2018). Baseline levels of IL-22 may be predictive of response to AhR therapy. A low level of IL-22 may predict a higher response rate to AhR therapy.

Other baseline measurements related to AhR biology may correlate with response to AhR agonists. Diagnostics for clinical use that may enhance or improve a response or a response rate to AhR agonist therapy are described and contemplated.

In a first aspect, a method for preventing, reducing or mitigating adverse events prior to or during treatment of a subject with an AhR agonist compound is provided and now described.

Exposure to 2,3,7,8-tetrachlorodibenzo-p-dioxin, also known as TCDD or dioxin, may result in systemic, constitutive, chronic AhR activation. TCDD is unusual in its high potency and long half-life. TCDD is a one of the most potent known AhR agonists and it cannot readily be metabolized in humans. A single large exposure results in constitutive activation of the AhR for years leading to a number of possible toxicities, including chloracne, gastritis, hyperkeratosis, chronic obstructive pulmonary disease (COPD) and sebaceous gland involution.

Additional evidence that constitutive activation of the AhR may be problematic comes from mouse experiments with chronic dosing of TCDD, where mean arterial blood pressure was increased, but only starting after 15 days of exposure. Adverse events occur after longer exposure, suggesting that AhR signaling on its own is not toxic, but that constitutive, systemic activation for longer than a couple of weeks may result in toxicity

Although the AhR is highly conserved across species, the lethal dose for 50% of a group of animals (LD50) values for TCDD exposure varies over 5000-fold among species, from 1 μg/kg in guinea pigs to >5000 μg/kg in hamsters. Metabolism of particular AhR agonists can be highly variable and species dependent. In mice, the magnitude of response to TCDD depends on the AhR allele with an Ala-to-Val substitution at codon 375 reducing affinity for TCDD and subsequent xenobiotic metabolic enzymes (Mimura 2003).

Polymorphisms in the genes or regulatory elements in the AhR pathway or interacting pathways may alter the response to or metabolism of AhR agonists. A number of known polymorphisms in the AhR have been shown to result in a loss of CYP1A1 induction (Wong 2001). Other polymorphisms in either the AhR or HIF signaling pathways may contribute to adverse events from administration of AhR agonists.

Continuous activation of AhR either due to a genetic polymorphism, continued administration of AhR agonists or a lack of metabolism of AhR agonists may cause sequestration of ARNT (also known as HIF-1β). ARNT plays an important physiological function from its role as a sensor of hypoxia, and deletion of the gene encoding ARNT (HIF-1β) is lethal. ARNT (HIF-1β) is the canonical binding partner for AhR and HIF-1α and HIF-2α, and sequestration of ARNT by one pathway may reduce the functionality of the other pathway (Vorrink et al., International Journal of Molecular Sciences 15(8):13916-13931 (2014)). ARNT (HIF-1β) binds HIF-1α or HIF-2α to form activated hypoxia inducible factor HIF1 or HIF2 respectively and which are the crucial transcription factors that mediate response to hypoxia. It is possible that constitutive activation of the AhR may result in sequestration of ARNT (HIF-1β) and an impaired response to hypoxic conditions which could lead to adverse events.

A recent publication (Masaki 2021) demonstrates the interplay between AhR and HIF signaling and shows that plasma levels of AhR agonists correlate with severity of pulmonary arterial hypertension (PAH). In PAH, it is unknown whether AhR agonists in the plasma are causative or correlated. The paper shows that in rats, both hypoxia and potent systemic AhR activation are necessary in the bone marrow and non-bone-marrow derived cells followed by normoxia to cause PAH-like disease. However, many patients with PAH have areas of the lung that are hypoxic due to inflammation and/or vasoconstriction. In these lung areas, HIF signaling could interfere with AhR signaling by sequestering ARNT (HIF-1β) and subsequent CYP1A production leading to impaired local metabolism of AhR agonists and hence higher levels of these agonists.

Alternatively, a flood of AhR activation on its own may overwhelm the available ARNT available. In the absence of ARNT (HIF-1β), agonized AhR (and to a lesser extend unagonized AhR) can interact with RelA and RelB and other proteins in non-canonical AhR signaling pathways resulting in expression of IL-8, IL-6 and various chemokines (Vogel 2007b). Other noncanonical signaling not-requiring ARNT (HIF-1β) can result in degradation of the estrogen receptor, downregulation of CYP1A1 and induction of various chemo-attractants (Lamas 2018). This non-canonical signaling may result in very different physiological effects compared to typical AhR signaling.

The link between AhR activation, endothelial/vascular expression of CYP1A1, high blood pressure and observed clinical adverse events such as reversible pulmonary arterial hypertension and myocardial ischemia suggest a possible causal link. Too much oxygen (hyperoxia) induces CYP1A1 in the lung and liver of wild-type B6 mice but not in AhR knock-out mice (AhR−/−) whereas too little oxygen (hypoxia) blocks induction of CYP1A1 by TCDD (Gradin et al., Molecular and Cellular Biology 16(10):5221-5231 (1996)); Vorrink et al., International Journal of Molecular Sciences 15(8):13916-13931 (2014)).

The mechanism for increase in arterial blood pressure following TCDD exposure was dependent on the presence of CYP1A1, which was shown to be expressed on aortic endothelium (Kopf 2010). It is not known whether CYP1A1 is having an active role in the process by increasing the metabolism of a substrate, or by some other mechanism. It is possible that CYP1A1 KO leads to increasing amounts of AhR agonists which could interfere with HIF signaling by sequestering ARNT (HIF-1β).

In some embodiments, the present methods are related to clinically advantageous companion diagnostics taken prior to the use of AhR agonists as a means to prevent adverse events, including but not limited to PAH.

In some embodiments, methods to prevent or mitigate adverse events may not require a companion diagnostic. In these cases, learnings from prior research or clinical may inform strategies that are expected result in a safer treatment with AhR agonists without the need to administer a diagnostic prior to beginning therapy. One such example is a strategy that enables the reduction of systemic exposure to a AhR agonist. This may be achieved by administering a compound that increases the metabolism of a AhR agonist prior to beginning therapy.

In other embodiments, the methods of monitoring for chronic, constitutive and/or systemic AhR signaling during AhR agonist therapy to reduce the risk of developing adverse events and enable interventions that prevent or mitigate adverse events in order to make this class of drugs safer or more tolerable.

Methods for motoring for early signs/symptoms of PAH or other cardiovascular AEs during AhR therapy are also desired.

In some embodiments, methods to monitor for chronic, constitutive and/or systemic activation of the AhR or for other early signs of the development of adverse events at the earliest stages are provided to prevent and mitigate adverse events during AhR agonist therapy.

Tryptophan and related indoles are the key starting materials for the eventual metabolism/catabolismand microbial production of AhR agonists. Reduced levels of these indole-derived AhR agonists results in less AhR activity. Changes in AhR biology that can be detected in patient biologic samples are predictive of response to therapy with AhR agonists or even AhR antagonists. These diagnostics can be utilized to predict response to AhR agonist or AhR antagonist therapy.

In one embodiment, methods for enhancing response to the administration of AhR agonists entail diagnosing the patient's baseline total AhR activity. Patients with low total baseline AhR activity are the most likely to respond to AhR agonist therapy. Those patients with the greatest baseline AhR activity are less likely to respond to AhR agonist therapy.

For example, plasma and stool analysis of ulcerative colitis patients who were treated with indigo naturalis may be used diagnostically. Baseline stool samples may be processed, for example, as reported in Lamas (Lamas et al., Nature Medicine 22(6): 598-605 (2016)) and tested in the AhR-luciferase assay (sold by Indigo Bioscience). Plasma samples may be processed as reported in Masaki 2021 and tested at a dilution of 10% in the AhR-luciferase assay. Total baseline AhR activity may be compared to positive control and to healthy control samples.

Those patients with the lowest quartile of baseline AhR agonists activity may be the most responsive to therapy with the AhR agonist mixture, indigo naturalis. Specifically, these patients may respond faster, more durably and with greater magnitude with regard to reductions in calprotectin, reduction of total and partial Mayo score, endoscopic activity score and histological scoring than the patients in the highest quartile of baseline AhR activity.

Thus, a diagnostic is envisioned where baseline stool or plasma is taken from patients prior to treatment with an AhR agonist as a means to evaluate whether AhR agonist therapy is a good treatment option.

In another embodiment, methods for enhancing responses the administration of AhR agonists entail measuring a patient sample for specific AhR agonists. These AhR agonists may be endogenously made by the body, made by the microbiota or dietary in nature.

AhR agonists can be measured in baseline patient samples via HPLC. Agonists may include one or more of the following or derivatives thereof: 3-hydroxy-indole, 3-methyl indole, astaxanthin, canthaxanthin, cinnabarinic acid, 3,3′diindolylmethane (DIM), diosmin, 6-formylindolo [3,2-b] carbazole (FICZ), indigo, indirubin, indole, indole-3-acetic acid (IAA), indole-3-aldehyde (IAld,), indole-3-acetaldehyde (IAAld), indole [3,2-b] carbazole (ICZ), indoxyl-3-sulfate (I3S), 2-(1′H-indole-3′-carbonyl)-thiazole-4-carboxylic acid (ITE), indole-3-acetonitrile (I3AC), indole-3-carbinol (I3C), kynurenine, kynurenic acid, luteolin, malassezin, myricetin, quercitin, resveratrol, tamarixetin, tangeritin and/or xanthurenic acid.

Patients with lower levels of one or more of the AhR agonists are more likely to respond to subsequent administration of AhR agonists. The therapeutically administered AhR agonist may be the same or different than the AhR agonist detected in the samples.

In another embodiment, methods for enhancing responses to the administration of AhR agonists entail measuring a patient for baseline plasma tryptophan levels. It is recognized that increased tryptophan metabolism is associated with activity in inflammatory bowel disease (Nikolaus et al., Gastroenterology 153(6):1504-1546 (2017)). It is believed that because tryptophan is the starting material for many endogenous AhR agonists that low tryptophan levels are a surrogate for lower AhR activity. Plasma levels of tryptophan have been shown to correlate with disease severity in Crohn's disease and ulcerative colitis.

Plasma tryptophan levels have been explored as a biomarker for response to infliximab and to vedolizumab (Nikolaus et al., Gastroenterology 153(6):1504-1546 (2017)). It was shown that infliximab responders have a sustained increase in plasma tryptophan levels compared to non-responders. This effect was not seen with vedolizumab. Plasma tryptophan levels have not been investigated as a predicative marker for AhR therapy.

Without being bound by any particular theory, baseline plasma tryptophan levels in IBD patients, specifically UC patients, may be predictive of response to AhR therapy. By segregating IBD patients based on the levels of plasma tryptophan, a predictive diagnostic can be developed that improves the odds and/or magnitude of response to AhR therapy.

Specifically, tryptophan and similar small molecules can be measured via ELISA, HPLC, UPLC, bioluminescent, coulometric or fluorometric assays.

It has also been shown that the plasma kynurenic acid/tryptophan ratio along with expression of various metabolic enzymes in the tryptophan metabolic pathway correlate with endoscopic subscore in UC (Sofia 2018). Specifically, plasma levels of kynurenic acid and picolinic acid correlated with mayo endoscopic subscore. As the kynurenic acid/tryptophan ratio is normalized to tryptophan it provides a more standardized metric of the kynurenine biosynthetic pathway than kynurenic acid alone. Downstream KYN metabolites such as kynurenic acid, quinaldic acid, anthranilic acid, xanthurenic acid, 3-hydroxykynurenine, 3-hydroxyanthranilic acid, a-amino-B-carboxymuconate-E-semialdehyde, quinolinic acid, A-aminomuconate-E-semialdehyde, picolinic acid and/or glutaryl CoA can also be measured alone or as a ratio to tryptophan or other related molecules.

In addition to cellular biosynthetic or catabolic processes, the human microbiota produces a number of AhR agonists, many of them made from tryptophan or indole precursors. It has been demonstrated that high bacterial species diversity correlated with high serum tryptophan levels (Nikolaus 2017). Those patients with lower species richness or lower levels of specific or total microbes known to produce AhR agonists may have decreased AhR signaling and may be more responsive to AhR agonist therapy. Therefore, methods to detect microbial richness and/or specific microbes known to produce AhR agonists may be utilized as a diagnostic prior to administration of AhR agonists.

Specifically, DNA or RNA can be extracted from patient samples, such as the stool, colon, intestine, or skin and the 16S rRNA amplicon sequencing can be performed on gut/skin samples in order to identify, classify and quantify bacterial species. In addition to the overall species richness which can be described by the number of distinct amplicons, specific bacterial species and strains can be identified that are known to produce AhR agonists. These species include but are not limited to; Bacillus alvei, Bacteroides sp., Bacteroides thetaiotaomicron, Citrobacter sp., Clostridium novyi, Clostridium limosum, Clostridium tetani, Corynebacterium acnes, E. coli, Flavobacterium sp., Fusobacterium sp., Haemophilus influenza, Kleibsella planticola, Kleibsella pneumonia, L. reuteri, Lactobacillus taiwanensis, Lactobacillus bulgaricus OLL1181, Providencia stuartii, Shigella flexneri and/or Vibrio cholera or any other related species or other species capable of making AhR agonists. A similar approach can be used separately or in combination to identify, classify and quantify fungi or viruses that produce AhR agonists. Specific examples of fungi may include subspecies of Malassezia yeast such as M. globosa CBS7966 and M. restricta known to produce indirubin). In one embodiment, lower levels of M. globosa strains on the skin would predict an enhanced response to AhR therapy.

Enzymes that metabolize tryptophan or related metabolic/catabolic products may be predictive of response to AhR agonists. These enzymes may be involved in the kynurenine pathway that starts with tryptophan. Differential activity in these enzymes may result in a reduction of specific AhR agonists and may be predictive of response to AhR agonist therapy. These enzymes may be measured via mRNA expression, protein levels or enzymatic activity. Specifically, these enzymes may include; tryptophan 2,3-dioxygenase-2 (TDO) and/or indoleamine 2,3-dioxygenase-1 (IDO) or any other enzyme or combination of enzymes in the kynurenine pathway.

Tryptophan may also be used in the body to generate serotonin and melatonin. Thus, levels of AhR activity may correlate with serotonin or melatonin, which can be measured in the plasma or other biological samples and may be used as a predictor of response to AhR agonist therapy.

CARD9 single nucleotide mutations have a linkage to inflammatory bowel disease. Specifically, patients with the following CARD9 mutations rs10870077, rs4077515, rs10781499 are at increased risk for IBD. As CARD9 has a role in AhR biology, these patients may be differentially responsive to AhR agonist compared to patients with more common CARD9 alleles.

Compared to healthy controls, IBD patients have reduced levels of plasma IL-22 (Sakemi 2020). Baseline cytokine expression may be predictive of response to AhR agonists. Specifically, IL-6, IL-17A, IL-23, IL-10 and IL-22 which are implicated in CARD9 biology and can be regulated by AhR signaling and may be measured alone or in combination with other metrics. For example, the tryptophan/IL-22 ratio may be predictive of response to AhR therapy.

Other AhR regulated genes include anti-microbial proteins or peptides. Specifically, these include; S100 proteins (S100A7, S100A8, S100A9), late cornified envelope proteins (LCE1A, LCE3A, LCE3E), SLP1, LL37, SKALP, hBD2, regenerating islet-derived protein 3 gamma (REG3G), regenerating islet-derived protein 3 beta (REG3B). Low levels of these proteins are suggestive of low AhR activity and may be a suitable biomarker for predictive response to AhR agonist therapy.

Still other AhR regulated genes includes proteins involved in barrier function including ZO-1, involucrin, occludin, tricelluin, claudin-1, claudin-5. Lower expression of these genes or gene products may be a suitable biomarker predictive of response to AhR agonist therapy.

The methods herein enable diagnostics that can be used to enhance the response rate for patients prior to starting AhR therapy or alternatively not to start AhR therapy due to the low likelihood of clinical response.

Also contemplated are methods for preventing adverse events prior to administration of an AhR agonist. While some AhR agonist-based compounds may have a suitable profile for the treatment a disease, adverse events are more likely to occur if (1) the AhR pathway is constitutively and/or chronically turned on 2) the AhR agonists are systemically bioavailable 3) the AhR agonists are not readily metabolized and/or 4) and the agonists are more active than desired. Methods for preventing AEs prior to or during AhR therapy entail diagnosing either genetic, epigenetic or environmental factors that may lead to prolonged, systemic or overly robust activation of the AhR. Other methods for preventing AEs prior to AhR therapy may rely on careful selection of starting dose andor use of a metabolic induction dose of a drug capable of inducting the therapeutics AhR agonist and may not require the use of a diagnostic.

Polymorphic genes related to AhR signaling, HIF signaling, AhR agonist metabolic activity, or related to the development of specific diseases, such as pulmonary arterial hypertension may increase the risk of developing adverse events in combination with AhR agonist therapy.

In some embodiments, genetic changes can be identified by polymerase chain reaction, mRNA expression, proteins expression or binding or enzymatic activity. The changes may be caused by a single nucleotide polymorphism (SNP), rearrangement, translocation, deletion, repeat or any other change to the DNA. The changes may result in a loss of function, change of function, increase of function, change in expression, alteration in regulation, alteration in protein binding or any other change that might impact the development of adverse events.

Changes may also be epigenetic in nature, resulting in a change in the way a specific gene or group of genes is regulated. Certain environmental effects are known to have a prolonged effect on how some genes are regulated. These changes may be detected by looking at chromatin immunoprecipitation or by evaluating the amount of histone modification, DNA methylation or expression of small non coding RNAs.

Environmental activities may also influence the eventual development of adverse events in combination with AhR agonist therapy. Activities which may lead to hypoxia or HIF signaling in the lung in combination with AhR agonists may have negative consequences. Alternatively, environmental factors which alter the metabolism of AhR agonists or increase the amount of alternative AhR agonists may have negative consequences in combination with AhR agonist therapy.

Diagnostics looking at the above can inform treatment decisions with AhR agonists prior to or after beginning therapy with AhR agonists. The resulting information has utility in being able to prevent or mitigate or inform about the risk of development of possible adverse events.

Long-term, systemic constitutive activation of the AhR may result in toxicities, which can present as side effects or adverse events. This may happen due to unsafe drugs, unsafe doses, unsafe dosing, concomitant therapies, concomitant activities or other genetic, environmental or metabolic factors. Unsafe drugs may be those, which result in systemic exposure to the parent compound or an AhR stimulating metabolite that is not metabolized into an inactive compound. Unsafe doses or dosing may result in systemic and/or chronic activation of the AhR without an opportunity for this pathway to relax.

Even limited exposure to high levels of AhR agonists may result in adverse events and/ortoxicity. Many AhR agonists have other activity at high levels. For example, tapinarof is known to be cytotoxic at level above 5 uM. So even acute high levels of AhR exposure may cause cell damage leading to acute inflammation. Other AhR agonists can induce the expression of multiple liver enzymes that could be interpreted as signs of liver damage. This may occur after the first dose, if there are high levels in the liver.

In one embodiment, a diagnostic would evaluate DNA polymorphisms in the AhR or in ARNT. Specifically, there are known polymorphisms at codon 517, 554 and 570 of the AhR. Experiments have shown that Lys554+Ile570 and the Lys554+Ile570+Ser517 variants failed to properly induce CYP1A1 upon exposure to TCDD (Wong et al., Biochemical and Biophysical Research Communications 288(4):990-996 (2001)) and these individuals may also have inhibited metabolism of some AhR agonists, specifically those which are metabolized by CYP1A1. SNP rs2228099 of the ARNT has been associated with essential hypertension and may also be of utility in predicting adverse events (AEs) in combination with AhR agonist use (Polonikov et al, Pharmacogenetics and Genomics 27(2):57-69 (2017)).

In another embodiment, a diagnostic that evaluates a DNA polymorphism in CYP450 enzymes or xenobiotic metabolic enzymes regulated by XREs, such as CYP1A1, CYP1B1 and/or CYP1A2, is contemplated. For example, there are known polymorphisms in CYP1A1 such as SNPs: rs4646903 (also known as CYP1A1*2A or as 6235[T/C]) which maps 3′ of CYP1A1, and rs1048943, a non-synonymous SNP that results in an Ile462Val substitution. Other known polymorphisms in CYP1A1 occur in codons 286 and 461. The CYP1A1 polymorphism Val462 has been linked to increased rates of lung cancer. This polymorphism is also found in higher frequency in Asian than Caucasian populations. Known polymorphisms in CYP1A2 include the 163C>A polymorphism (rs762551). Known polymorphisms and mutations in CYP genes or related XREs are incorporated by reference (Saito et al., Cytokine 136: 155264 (2002)). Specifically, SNPs-1061, -1035, -1020 and -947 in the XREs for CYP1A1 and -1222, -376 and -265 in the XRE for CYP1B1.

In another embodiment, a diagnostic that evaluates polymorphisms near the XREs that modulate AhR function is provided. For example, rs2470893 is a known SNP located 196 base pairs from a CYP1A1 promoter XRE (Liu et al., Drug Metabolism and Disposition 46(9): 1372-1381 (2018)).

In another embodiment, a diagnostic that evaluates polymorphisms in the aryl hydrocarbon receptor repressor that modulates AhR function is provided. For example, the Ala185Pro polymorphism.

In another embodiment, a diagnostic that evaluates polymorphisms in the gene BMPR2, which has been linked to the development of PAH, (Masaki et al. Proceedings of the National Academy of Sciences 118(11) (2004)), is provided. In a related embodiment, the CYP1B1 polymorphism N453S is evaluated in combination with polymorphisms in BMPR2.

In another embodiment, a diagnostic that evaluates polymorphisms in the gene HIF1α, specifically the AA genotype at rs12434438 is envisioned.

In one embodiment, the diagnostic creates a polymorphism risk score based on multiple polymorphisms indicative of the relative risk of taking an AhR agonist. This score may or may not be combined with other clinical, medical or diagnostic data in making a determination about the suitability of a particular AhR agonist or the dose or dosing of such agonist to treat a disease.

In another embodiment, a diagnostic evaluates a recombinant AhR with a known polymorphism of the AhR to directly bind an AhR agonist or to induce CYP1A1 expression. In the former case, recombinant AhR protein or simply cell lysate is bound to an antibody for the AhR and a solution of the AhR agonist is washed over and a shift in light scattering observed. Alternatively, a secondary antibody is used in an enzymatically linked immunosorbent assay (ELISA) based assay. Alternatively, recombinant AhR is added to ARNT along with the AhR agonist compound and an electrophoretic mobility shift assay is used to determine binding to DNA response elements or evaluated in the ability to induce CYP1A1 in Hepa-1 Group B mutant cells which express low endogenous AhR levels and do not normally express CYP1A1 in response to AhR agonists.

In another embodiment, a diagnostic that evaluates the ability of microsomes or cells biopsied from a patient to metabolize an AhR agonist is provided. In one example, the microsomes or cells are from the blood, skin, vasculature, liver, intestine, colon or lung are envisioned. In this example, the activity of these cells or microsomes is detected by evaluating their ability to metabolize an AhR agonist using a reporter cell line. Other similar types of readouts, including but not limited to coulometric or enzymatic or light scattering, could be used.

In another embodiment the chromatic architecture or DNA methylation status of the CYP1A1 promoter is investigated. In one case the DNA architecture of the CA1 and CA2 CYP1A1 promoter region is assessed via a nuclease accessibility assay as described in Vorrink 2014. A more closed promoter region would be expected to not be as inducible to activated AhR and may result in less CYP1A1 induction and a longer exposure to the AhR agonist therapy which could result in adverse events.

Concomitant medications or activities may serve to activate the AhR or to potentiate the effects of an AhR agonist. Concomitant medications that activate the AhR may include ketoconazole or omeprazole. Specific activities of concern that may result in AhR activation include dietary AhR agonist consumption such indole-3-carbinol containing vegetables like broccoli, smoking, which results in the exposure to polyaromatic hydrocarbons which are AhR agonists, exposure to AhR agonists in the form of pollutants, or exposure to hyperoxic conditions like when scuba diving.

Along similar lines, concomitant medications, drugs or foods which inhibit the metabolism of an AhR agonist could result in unsafe prolonged exposure to an AhR agonist. For example, indirubin is metabolized by CYP1A1, and known inhibitors of CYP1A1, such as grapefruit juice (bergamottin) and marijuana, including isolated compound from marijuana such as tetrahydrocannabinol (THC) or cannabidiol (CBD), may result in inhibited expression of CYP1A1 and higher and/or prolonged exposure to indirubin after administration.

Consumption of foods, drugs or supplements that increase the absorption or dissolution of an AhR agonists could increase systemic exposure. Specifically, a high fat diet or high alcohol could increase the absorption some AhR agonists and may be recommended against.

In another embodiment, patients judged to be at increased risk for the development of pulmonary arterial hypertension would be excluded from treatment with an AhR agonist or treated with a lower dose and/or dosing of an AhR agonist. Examples of increased risk patients may include those with a family history of heritable PAH, connective tissue disease, human immunodeficiency virus (HIV) infection, portal hypertension, congenital heart disease, schistosomiasis, or drugs or toxins associated with PAH including, but not limited to, methamphetamines, fenfluramine, aminorex, dexfenfluramine, benfluorex, dasatinib, and toxic rapeseed oil.

In another embodiment, laboratory or clinical diagnostic values related to cardiovascular function may form the basis of a diagnostic. These include systolic blood pressure (systolic BP) or mean pulmonary arterial pressure (mPAP). In other examples the laboratory values are echocardiographic parameters such as pulmonary arterial systolic pressure (PASP), left atrial dimension (LAD), left ventricle end-diastolic diameter (LVEDD), left ventricular ejection fraction (LVEF), inferior vena cava dimension, right atreial area (RA area), tricuspid annular plan systolic excursion (TAPSE) or tricuspid regurgitation pressure gradient (TRPG). In other cases the cardiovascular function would be focused on valvular abnormalities or valvular heart disease and evaluated via echocardiogram looking for aortic regurgitation, mitral regurgitation, valve thickening or restrictive valve motion. In some specific examples, the reference laboratory value is a measurement of PASP as estimated via echocardiography or as measured via right heart catheter prior to starting therapy or after 1, 3, 6, 12, 18, 20, or 24 months of continuous or discontinuous therapy with an AhR agonist.

In one embodiment a drug label for an AhR agonist would specifically exclude the concomitant use of CYP1A1, CYP1B1 and/or CYP1A2 inhibitors or medications or drugs with AhR agonist activity.

In another embodiment a drug label for an AhR agonist would specifically exclude the concomitant use activities or environmental factors that may result in hypoxia, such as scuba diving, mountain climbing, flying at high altitude or prolonged residence above a certain altitude such as above 7,000, 8,000, 9,000, 10,000 or 11,000 or higher feet.

In another embodiment a drug label for an AhR agonist would specifically exclude patients that have medical conditions leading to hypoxia, such as sleep apnea.

In another embodiment a drug label for an AhR agonist would specifically exclude consumption of a high-fat diet or the consumption of alcohol.

Any of the above methods, alone or in combination may form the basis of a diagnostic to prevent or mitigate the development of adverse events prior to or during use of an AhR agonist therapy.

In some cases, it may not be possible to appropriately mitigate or eliminate the development of adverse events prior to beginning AhR agonist therapy, in these cases diagnostics that monitor for the development adverse events before they occur or become serious are warranted. Methods for monitoring, reducing, and/or treating adverse events during administration of an AhR agonist are provided.

In one embodiment, a diagnostic for AhR systemic activation relies on the EC50 or another other reasonable amount of an AhR agonist needed for activity for the human AhR and the pharmacokinetic profile of the AhR agonist in the plasma, blood or tissue. A diagnostic may be used to ensure that the dose or dosing did not exceed a concentration (for example the EC50) able to activate the AhR for a given period of time. For example, in the case of indigo, plasma levels above 1 nM, 10 nM, 50 nM, or 100 nM may serve as a cutoff. In the case of indirubin, plasma levels of 0.1 nM, 1 nM, 10 nM or 25 nM could serve as a cutoff. An amount of time for which the patient might not exceed this threshold concentration may be 1 day, 1 week, 1 month, 3 months or some other amount of time.

Constitutive activation of the AhR may result in a reduction of free ARNT and thus a reduction of the heterodimeric transcription factor hypoxia induced factor (HIF). In these circumstances genes or proteins regulated by HIF and process regulated by these proteins may be inhibited. For example, there may be a reduction in erythropoietin in the blood. Alternatively, there could be a reduction in the number of red blood cells, a process regulated by erythropoietin and this could be evaluated by hematocrit. Measurements of leukocytes, hemoglobin, hematocrit of thrombocytes may correlate with AhR activity. In one embodiment, a hematocrit that drops 10% from baseline may be indicative of too much or constitutive systemic AhR exposure.

In another embodiment, a diagnostic test measures the ability of cells or microsomes from a patient using the ethoxyresorufin-O-deethylase (EROD) assay, which is an assay for measuring the activity of xenobiotic metabolizing enzymes such as CYP1A1 and CYP1A2 in their ability to mediate deethylation of the 7-ethoxyresorufin substrate into resorufin (https://protocolexchange.researchsquare.com/article/nprot-3473/v1). Increased activity in this assay suggests more expression of these enzymes and greater exposure to an AhR agonist. For example, measuring significant EROD activity in PBMCs for an AhR which is supposed to be locally acting would be indicative of systemic exposure.

In another embodiment, a diagnostic that measures a blood level of an enzyme that is indicative of systemic AhR activation and/or liver toxicity is provided. For example, a diagnostic that measures a liver enzyme blood level, such as of alanine aminotransferase (ALT) or aspartate aminotransferase (AST), is provided. In an embodiment, an ALT level elevated two-fold above baseline for longer than one week is indicative of the potential for development of an adverse event.

In another embodiment, a diagnostic that evaluates a gene or gene product regulated by XREs is provided. The gene can be for example CYP1A1, CYP1A2, CYP1A2 CYP19A1, IGFBP10, GSTP1 or any of the genes listed in Adachi et al., Toxicological Sciences. 80, 161-169 (2004). Combinations of these genes or gene products can serve as biomarkers with predictive value for the development of an adverse event AhR therapy. For example, a 50% increase in both CYP1A1 and AhRR from baseline in PBMCs may be a signal of too much systemic AhR agonist exposure.

The expression of metabolic enzymes such as CYP1A1 and CYP1A2 may increase the expression of metabolic products or decrease the expression of known CYP1A1 and CYP1A2 substrates. For example, CYP1A1 metabolism of tryptophan substrates may increase levels of serotonin. The levels of serotonin in the blood are normally extremely low. Therefore, a diagnostic evaluating serotonin or tryptophan, 5-hydroxy-L-tryptophan (5-HTP) or 5-hydroxyindoleacetuc acid (5-HIAA) levels in the blood or tissue could have diagnostic properties.

In a related example, a substrate of CYP1A2 is melatonin. Therefore, looking for melatonin at lower-than-expected levels in the blood, urine or saliva could be diagnostic of systemic AhR activation.

Cardiovascular monitoring may identify adverse events during treatment with an AhR agonist before they patients become symptomatic. The findings from continued cardiovascular monitoring may form the basis of a risk evaluation and mitigation strategy (REMS). In the case of a AhR agonist drug REMS program, patients must enroll in and comply with ongoing cardiac monitoring requirements which may include echocardiogram monitoring and/or a routine safety questionnaire. In one embodiment of the AhR REMS program, patients must receive counseling about the potential risks of the AhR agonist, agree to an echocardiogram every six months and can only get their prescription filled by a pharmacy participating the in the AhR REMS program in order to continue to receive their prescription. The prescribing prequalified physician would evaluate the echocardiogram every 6 months to look for adverse signals such as a changes in pulmonary arterial pressure or valvar heart disease or new valvular abnormalities. The physician would evaluate the risk and pass their recommendations onto a central computerized REMS program that would relay the decision to the patient and associated pharmacy that would administer or not administer the patient's prescription accordingly.

Details on REMS programs have been described with the use of sodium oxybate for the treatment of narcolepsy (U.S. Pat. Nos. 6,045,501, 6,315,720, 6,561,976, 6,561,977, 6,755,784, 6,869,399, 6,908,432, 7,141,018, 7,874,984, 7,959,566, 8,204,763, 8,315,886, 8,589,188 and 8,626,531), thalidomide for the treatment of multiple myeloma (U.S. Pat. Nos. 7,668,730, 7,765,106, 7,765,107, 779,717, 7,895,059, 8,263,650, 8,457,988, 8,589,182, and 8,731,963) and fenfluramine for the treatment of epilepsy (U.S. Pat. No. 10,950,331). Disclosure related to the REMS programs in these publication is incorporated by reference herein.

An AhR agonist REMS program may rely on use of a central controller which is a preprogrammed centralized database connect to the internet or other manner of telecommunication capable of analyzing data and communicating findings to physicians, pharmacies and patients. In some cases, the central controller would not allow for the initial or renewed prescription for an AhR agonist to be filled unless a series of conditions are met by an authorized user inputting appropriate data. For example, one conditions may include the timely input of data from routine echocardiograms demonstrating no increased cardiovascular risks.

Any of the above methods, alone or in combination may form the basis of a diagnostic to prevent or mitigate the development of adverse events after beginning use of an AhR agonist therapy.

In any of the above aspects and embodiments, the diagnostic information can be utilized by the patient, treatment protocol or a healthcare provider to modify the dosage or dosing schedule or can be used to restrict a possible patient from taking a particular AhR agonist compound, or given a different AhR agonist compound that one originally considered. In some embodiments, a patient may be recommended to reduce the dosing of AhR agonist compound to three times daily, twice daily or once per day, once for 5 days and then 2 days off, every other day, for 1 week and then 1 week off, for one month and then one week off or any other intermittent dosing regimen that may prevent or mitigate the development of adverse events. Alternatively, they may be recommended to reduce the daily dose by 25%, 50%, 75% or any other amount.

In other embodiments, the subject is given concomitant medications that may result in the increased metabolism of the AhR agonist or given a medication that acts as a local AhR antagonist. For example, a patient could be given a drug that is known to increase the activation of a CYP1A1 in the lung to prevent the development of PAH during AhR agonist therapy. Alternatively, they may be recommended a diet or consumption mechanism to reduce systemic exposure such as by consuming the AhR agonist with a meal, a glass of water, a glass of milk or in the absence of a high fat or high alcohol diet.

In any of the above embodiments, the diagnostic has predictive value for detecting, preventing or reducing the development of an adverse event or side effect, including but not limited to folliculitis, essential hypertension, pulmonary arterial hypertension, pneumonitis, respiratory failure, myocardial ischemia, myocardial infarction, pericarditis, pleuritis, peritonitis, pneumonitis, chest pain, abdominal pain, diarrhea, vomiting, intussusception, joint pain, back pain, liver damage, changes in liver enzymes, infection, peripheral neuropathy, headache, pancytopenia, and/or leukopenia.

In any of the above embodiments, various interventions can be implemented after a diagnostic result is obtained to minimize the severity of a possible adverse event or side effect. For example, a physician could administer antioxidants, angiotensin II receptor blockers, prostacyclin analogs or receptor agonists, endothelin receptor antagonists, PDE-5 inhibitors or soluble guanylate cyclase stimulators to minimize or prevent the development of PAH.

In any of the above embodiments, the diagnostic information can be used to adjust the dose or dosage of a drug containing an AhR agonist. The information can be used to select a patient for additional monitoring or testing to minimize the chance of an adverse event. The information can be used to include or exclude a patient from clinical study or from eligibility of receiving the drug. The information can be used on the label of a drug product. The information can be used to adjust concomitant medications, supplements, foods or drinks, diets, habits or activities that a patient should or should not participate in, for example, exposure to hypoxic conditions, ingestion or inhalation of tobacco or marijuana or cannabinoid-based products or use of hormonal birth control.

If high exposure to an AhR agonist or systemic activation of the AhR pathway or poor metabolism of an AhR agonist is detected, the methods herein contemplate intervening to prevent or reduce toxicity. If a method for determining which patients or activities would be problematic with a given AhR agonist are known, then a treatment strategy with modified dosages or dosing could be enacted. Alternatively, such patients could be recommended against taking AhR agonists.

The methods described herein serve the basis for a set of diagnostics which inform treatment decisions prior to or during AhR agonist therapy. They have utility in increasing response to therapy and in preventing and mitigating adverse events.

Also contemplated are strategies to mitigate possible adverse events prior to administration of an AhR agonist, where the strategy or method does not require a diagnostic. For example, a strategy of pre-administration of a compound prior to therapeutic dosing with an AhR agonist without the need of a diagnostic test may reduce and/or prevent the development of adverse events.

In one embodiment, the strategy uses a subtherapeutic dose of the therapeutic AhR agonists as a means of turning on the local metabolism. This strategy serves as means of increasing the rate of first pass metabolism which minimizes systemic exposure to the subsequent therapeutic dose of the AhR agonist.

Metabolic enzymes like CYP1A1 have half-lives measured in hours or days, so this strategy may be utilized with AhR agonists to be given as once daily therapies. The same strategy would have an impact on AhR agonists administered every other day, twice daily or three times daily.

This strategy may utilize a dose that is 0.1%, 1%, 5%, 10%, 20%, 30%, or 50% of the therapeutic dose. It may be administered 1 week, 3 days, 2 days, 1 day, 12 hours, 8 hour, 4 hour, 2 hours, 1 hour, immediately before or concurrently with the therapeutic dose. In an embodiment, the administering increases local AhR metabolic rate.

This strategy may result in low or no systemic exposure to the lead in dose and would by definition induce an enzyme or enzymes capable of metabolizing the subsequently administered AhR agonist locally. The lead in dose may or may not have any therapeutic effect on its own. The lead in dose may or may be an AhR agonist itself.

This strategy may result in low or no systemic exposure of the therapeutically administered AhR agonist. This strategy may result in a lower Cₘₐₓ, than would be measured without employing this strategy. The Cₘₐₓ of the therapeutic AhR agonist in the plasma or in tissues may be reduced by 100%, 99%, 90%, 75%, 50%, 25% or 10% or any other clinically meaningful amount.

This strategy may result in a lower area under the curve (AUC) of the therapeutically administered AhR agonist in the plasma or tissues, than would be measured without employing this strategy. The reduction in AUC may be evaluated from 0 to 24 hours and may be reduced by 100%, 99%, 90%, 75%, 50%, 25% or 10% or any other clinically meaningful amount.

This strategy may result in a lower liver exposure and therefore lower production of liver enzymes. Some liver enzymes such as ALT and AST can be produced when the liver is damaged or stressed and high levels of these, and related biomarkers, may be viewed as adverse events. This strategy may result in a reduction in plasma ALT and/or AST by 100%, 99%, 90%, 75%, 50%, 25% or 10% or any other clinically meaningful amount compared to a strategy that does not utilize a run-in dose. This decrease may be observed 1 day, 2 days, 3 days, 1 week, 1 month, 2 months, 3 months, 6 months, 9 months or 1 year following the beginning of AhR agonist therapy.

This strategy may be restarted as necessary if the patient misses a dose. This strategy could be restarted if a patient misses, 1, 2, 3, 5, or 10 doses or goes without a dose for 24, 48, 74, 96 hours, 1 week, 2 weeks, 3 weeks, 4 weeks. The strategy could be restarted based on the results of any of the diagnostic tests enclosed within.

### III. Examples

The following examples are illustrative in nature and are in no way intended to be limiting.

### Example 1

### Level of Total Baseline AhR Agonist in Plasma

Prior to starting therapy with a derivative of indol-3-acetic acid for the treatment of moderate-to-severe Crohn's disease, a plasma sample is collected in the morning following an overnight fast. The plasma is tested using the AhR-luciferase assay at 10% plasma as described in Masaki 2021. The patient has 95% reduced AhR-luciferase activity compared to the mean activity level of healthy volunteers. Compared to matched plasma from moderate-to-severe Crohn's disease patients, the plasma has just 25% of the average activity. Thus, the patient has dramatically reduced plasma AhR agonist activity levels compared to healthy control plasma and as compared to matched moderate-to-severe Crohn's disease patients. Based on clinical biomarker data, patients with less than 50% of the relative AhR-luciferase activity in their plasma compared to moderate-to-severe Crohn's disease plasma are more likely to meet the primary clinical endpoint of clinical remission (defined as a Crohn's Disease Activity Index of less than 150) by week 4 when treated with derivatives of indol-3-acetic. The gastroenterologist writes a prescription for the derivative of indol-3-acetic acid therapy based on these data, as the patient is more likely than other similar patients to respond to therapy.

### Example 2

### Level of Total Baseline AhR Agonists in the Stool

Prior to starting therapy with a delayed-release tablet containing an indirubin prodrug for the treatment of refractory moderate-to-severe ulcerative colitis, a set of three stool samples are collected on three consecutive days at the same time at home and frozen. The samples are homogenized in saline solution, processed to remove any particulate matter and sterile filtered and then frozen. The samples are then added in at 10% of the total volume to a AhR-luciferase assay. The average relative AhR-luciferase activity compared to Mayo-score matched refractory moderate-to-severe ulcerative colitis is 800%. Clinical data from prior studies has suggested that patients with the highest relative AhR-luciferase activity in the stool are the least likely to respond as the colon is likely already being stimulated with AhR agonists. Thus, the patient or care provider decides to try an alternative therapy.

### Example 3

### Level of AhR Agonists in the Urine

Prior to starting therapy with a delayed release gel cap containing a suspension of indigo for the treatment of ulcerative colitis, the total amount of indirubin and indigo in the urine are determined (Adachi et al., Toxicological Sciences 80(1):161-169 (2001)) from 6 frozen urine samples taken at waking and before bed over 3 days. Healthy urine contains an average of 0.22 uM of indigo and 0.19 nM of indirubin. However, this patient contains less than 0.01 uM of combined indigo and indirubin. Previous research suggests that less than 0.10 uM of either indigo or indirubin makes the candidate more likely to respond to therapy. Thus, the patient is started on the indigo therapy.

### Example 4

### Level of Cytokines in the Serum

Prior to considering use of an oral delayed-release dosage form containing a solid dispersion of indigo for the treatment of ulcerative colitis, a baseline blood sample is taken. The level of IL-22 in the serum is determined via ELISA. The patient has levels below 1 pg/ml. The mean level of IL-22 for UC patients was determined to be 16 pg/ml (Sakemi et al., Journal of Human Genetics 47(8):419-444 (2020)). Thus, the patient has levels lower levels of IL-22 than the typical UC patient, and this low level of IL-22 suggests that drugs which induce IL-22 expression are more likely to be effective. Thus, the patient may respond better to AhR therapy for their UC and is treated with the oral indigo product.

### Example 5

### Level of Specific Bacteria and Bacterial Species Richness in the Stool

Prior to starting therapy with a suppository containing indirubin for the treatment of proctitis, a stool sample is taken and the level of specific species and major bacteria phyla were determined by 16s-rRNA sequencing. The level of lactobacilli, a phyla known to produce AhR agonists was 100 cfu/g of stool compared to healthy controls which have an average of 30,000 cfu/g. Specifically, Lactobacillus bulgaricus OLL1181, a bacterium known to produce AhR agonists, was not detected in the stool compared to healthy controls which have an average of 1,000 cfu/g. In addition, bacterial species richness as measured by unique amplicon sequence variants was determined. The patient had 10% of the average species richness compared to typical UC patients. Based on the data, the patient has reduced levels of bacterial species and reduced levels of specific bacterial species that are capable of making AhR agonists and is more likely to respond to therapy with the indirubin suppository and begins therapy.

### Example 6

### Level of Cypia Enzymatic Activity in Colon Biopsies

During endoscopy to evaluate patient's ulcerative colitis severity, a biopsy of the inflamed colon is collected. The biopsy is frozen and then processed for the EROD assay to evaluate the level of CYP1A1 activity. The patient has very low levels (less than two standard deviations from the mean) of EROD activity in the colon biopsy compared to matched UC controls which suggests the patient has low levels of AhR activity and is more likely than those with high EROD activity be responsive to therapy with an AhR agonist. Thus, the patient's gastroenterologist decides to treat the patient with an AhR agonist.

### Example 7

### Response of PBMCs to AhR Agonist

Prior to starting therapy with a topical AhR agonist for the treatment of pustular psoriasis, a blood sample is collected. PBMCs are processed and sent out fresh for stimulation with and without the AhR agonist FICZ. After 24 hours, EROD activity is measured. The patient sample is highly responsive to FICZ as measured by high EROD activity compared to EROD activity from healthy normal PBMCs. The ratio of stimulated to unstimulated EROD activity may be indicative of baseline AhR activity and responsiveness to AhR therapy. Thus, a high ratio of stimulated to unstimulated EROD activity suggests that a patient may responsive to therapy with an AhR agonist. Based on the data, the patient's dermatologist prescribes the topical AhR agonist for the treatment of pustular psoriasis.

### Example 8

### Polymorphisms Linked to a Lack of AhR Agonists

Prior to starting therapy with indirubin ointment for the treatment of hidradenitis suppurativa, a cheek swab is taken and a patient is screened for inflammatory disease risk alleles related to AhR signaling including mutations in CARD9. The results show that the patient has a mutation in CARD9 (mutations rs10870077). Based on this result the patient may have impaired baseline tryptophan metabolism leading to impaired AhR signaling contributing to their disease and be more responsive the AhR agonist therapy. Thus, the patient is started on the indirubin therapy.

### Example 9

mRNA Levels of AhR Induced Transcripts

Prior to starting therapy with FICZ derivative cream for the treatment of hidradenitis suppurativa, a skin biopsy of an active nodule is taken. The biopsy has 5% of normal levels of expression of mRNA from matched HS patients for S100A7, a protein which is induced by AhR signaling. Thus, the patient has abnormally low levels of S100A7 which is indicative of reduced levels of AhR signaling, which may be related to low levels of AhR agonists in the skin. Thus, the patient is more likely to respond to therapy with an AhR agonist therapy and the patient is treated accordingly with the FICZ derivative cream.

### Example 10

### Tryptophan Level in the Skin

Prior to starting therapy with tapinarof gel for the treatment of plaque psoriasis, a skin biopsy from a plaque is taken. The biopsy has 2% of normal tryptophan levels from matched patients. Thus, the patient has abnormally low tryptophan which suggests may be related to low levels of AhR agonists in the skin and low baseline AhR signaling. Thus, the patient is more likely to respond to therapy with an AhR agonist therapy and the patient is treated accordingly.

Examples for how to prevent adverse events prior to starting therapy with an AhR agonist that require a diagnostic:

### Example 11

### Testing of AhR Agonist Metabolism in Microsomes Prior to Therapy

Prior to beginning therapy with oral meisoindigo for the treatment of rheumatoid arthritis, a physician orders a diagnostic to assess ability of the subject to adequately metabolize meisoindigo. Blood microsomes are extracted from the subject and incubated in medium containing 10 nM meisoindigo for 24 hours. The microsomes are removed via ultra-centrifugation and the remaining medium is added to a reporter cell line (such as product IB06001 sold by Indigo Biosciences) that expresses luciferase behind the promoter for human CYP1A1 and which can detect AhR agonist activity. After incubating for 12 hours, and addition of luciferin, the cell line produces the same amount of light as a control media containing 10 nM meisoindigo suggesting little to no meisoindigo was metabolized in the presence of the microsomes over 24 hours. A control sample of microsomes from a healthy normal individual typically gives off the same amount of light as a control solution containing 0.1 nM meisoindigo after 24 hours, suggesting that a healthy normal can metabolize 99% of the added meisoindigo over 24 hours. Based on the results of this diagnostic, the physician believes the patient has a defect making them a poor metabolizer of meisoindigo and recommends against the use of meisoindigo due to the possibility of development of adverse events.

### Example 12

### Testing for Induction of Cypiai in a Test Spot Prior to Therapy

Prior to beginning therapy with 0.02% indirubin ointment for plaque psoriasis, a physician applies the ointment to a 20 cm diameter test spot. The next day the physician takes a biopsy of the treated skin from this treated spot and a control biopsy from untreated nearby skin and preforms immunofluorescent staining for CYP1A1 and AhR protein and for DNA via DAPI co-staining. No more CYP1A1 protein is detected in the treated skin compared to the control skin. In the same biopsies, AhR nuclear translocation is seen in most epidermal cells in the treated but not the control skin. The physician is concerned that this patient has a defect in CYP1A1 expression which might lead to the inability to metabolize indirubin and which would lead it to bioaccumulate. Therefore, the physician writes a prescription for a 0.002% indirubin ointment rather than 0.02% indirubin ointment.

### Example 13

### Testing for the Ability to Metabolize an AhR Agonist Test Dose In Vivo

Prior to beginning therapy with indigo naturalis, a urine sample and plasma sample are collected. The patient is given a single capsule containing 1.5 g of indigo naturalis which is swallowed in office. 24 hours later the patient returns to the office and a new blood urine and plasma sample is collected. Normally patients who take a test dose of 1.5 g indigo naturalis are able to metabolize the indirubin contained within indigo naturalis into metabolites like indigo carmine so that 24 hours later less than 0.1 nM indirubin remains in the plasma and so a concomitant increase in indigo carmine (above 5 nM) is observed in the urine. At baseline, the results show 0.01 nM indirubin in the plasma and 0.2 nM indigo carmine in the urine. After 24 hours the level of indirubin in the plasma is 10 nM and the level of indigo carmine is still 0.2 nM in the urine. Thus, this patient has a defect in their ability to metabolize indirubin to indigo carmine and is not started on long-term indigo naturalis therapy.

### Example 14

### Sequencing for Polymorphs Linked to Poor AhR Metabolism

Prior to beginning therapy of indigo for the treatment of ulcerative colitis, a physician does a DNA test on a patient sample to sequence CYP1A1 to look for SNPs linked to defects in indigo metabolism. The subject has a polymorphism in CYP1A1 (T3801C), which is linked to poor indigo metabolism and is therefore recommended against using any AhR agonist metabolized by CYP1A1.

### Example 15

### PAH Risk Score Prior to Starting AhR Therapy

Prior to beginning therapy of an amorphous solid dispersion of indigo designed to give systemic exposure for the treatment of rheumatoid arthritis, a comprehensive PAH risk score is conducted. If the risk score is greater than 2, AhR therapy will not be started, if it is between 1 and 2 it will be started with additional monitoring and if it is below 1 it will be started with standard monitoring. The score is an average of multiple weighted components all scored between 0 and 2. The components comprise genetic risk (BMPR2, AhR, CYP haplotypes), environmental components (dietary AhR intake, dietary CYP inhibitor intake, smoking status), age, sex, hypoxia exposure (airline travel, altitude of home, scuba diving, mountain climbing, sleep apnea, COPD, interstitial lung disease, exposure the environmental pollutants that are hypoxia mimetics) and current PAH risk factors (relatives with PAH, mean pulmonary arterial pressure and pulmonary arterial systolic pressure, total plasma AhR agonist levels). In this case the patient has a risk score of 0.55 and is considered lower risk for developing PAH so is started on AhR therapy for their rheumatoid arthritis.

### Example 16

### Cardiac Monitoring Prior to AhR Agonist Administration

Prior to beginning therapy with an oral suspension containing ITE for the treatment of lupus a physician must perform cardiac monitoring prior to treatment according to the label of the drug. In the case of this patient, echocardiogram identifies evidence of valvular heart disease as indicated by moderate mitral regurgitation with an additional characteristic of valval heart disease, namely valve thickening. Based on these findings the patient is deemed to be at high risk for development a cardiac adverse event and is not eligible for treatment with the AhR agonist.

### Example 17

### Lead in Dose to Minimize Changes in Liver Enzymes

It was noted in clinical development that standard dosing with twice daily dosing of 50 mg of indigo in the form of a modified release tablet was leading to a 5× increase in ALT and AST 2 days following administration in 50% of patients. As such, a product label identified a clinical regiment whereby a lead in dose of 5 mg indigo is administered once daily for 2 consecutive days prior to beginning the 50 mg therapeutic dose. This dosing regimen did not produce any notable changes in ALT or AST levels 2 days following the therapeutic dosing.

### Example 18

### Lead in Dose After Taking a Break From Therapy

It was noted in clinical development that once daily dosing of delayed release indirubin was leading to headaches during the first week of therapy. A lead in dose of a CYP1A1 inducer, in this case the weak AhR agonist omeprazole in the form of a standard omeprazole delayed-release capsule containing 20 mg of drug, was shown to reduce the development of headaches during the first week of therapy by serving as a metabolic inducer and reducing the Cₘₐₓ of indirubin. The label for the delayed release indirubin indicates that patients should take a delayed-release capsule containing 20 mg omeprazole 12 hours prior to taking their first dose of delayed release indirubin. The label also instructs that if a patient should miss or stop taking their daily delayed release indirubin for 2 more or doses, that they should take a delayed-release capsule containing 20 mg omeprazole 12 hours prior to taking their subsequent dose of delayed release indirubin as a means to reduce the occurrence of adverse events like headaches.

Examples for methods to minimize or mitigate adverse events while undergoing therapy with an AhR agonist:

### Example 19

### Reducing Dose Due to Systemic Detection of Liver Enzymes

A patient with treatment-resistant ulcerative colitis is treated with delayed release-capsules containing 1 gram of indigo naturalis twice daily. Prior to beginning therapy, blood samples are taken and the blood level of ALT is determined using standard laboratory techniques. Weekly blood draws are scheduled for the first 6 weeks. After two weeks of therapy, blood levels of ALT are 5× normal levels. The diagnostic instructs to reduce the dose by half to 0.5 g per day. After two weeks blood levels of ALT have returned to normal and the patient is maintained at 0.5 g per day.

### Example 20

### Reducing Dose and Dosing Due to Accumulation of AhR Agonist in the Plasma

A patient with pouchitis is being treated with an oral suspension containing 10 mg indirubin twice daily. Plasma samples are taken weekly during the first 6 weeks of therapy and sent out for PK analysis for indirubin levels. At baseline the indirubin is undetectable. At week 1 indirubin levels are 5 ng/ml. At week 2 indirubin levels are 25 ng/ml. The physician reduces the dose to 2.5 mg once per day. At week 3 indirubin levels are 2 ng/ml. At week 4 indirubin levels are 3 ng/ml. At week 5 indirubin levels are 2 ng/ml. At week 6 indirubin levels are 3 ng/ml. The patient is responding well to therapy. Based on the leveling out of the plasma indirubin levels at a level that would not be expected to systemically activate the AhR the doctor maintains a dose of 2.5 mg/day to prevent the development of adverse events.

### Example 21

Reducing Dose and Then Discontinuing Patient Due to Increasing Levels of Systemic AhR Signaling by Detecting CYP1A1 mRNA in PBMCs

A patient with Crohn's disease is given 50 mg of indirubin in an immediate release tablet once daily. A diagnostic is set to evaluate the mRNA levels of both CYP1A1 and CYP1A2 in the blood using RT-PCR of peripheral blood mononuclear cell mRNA every two weeks. After two weeks of therapy, the level of CYP1A1 mRNA is increased 12× and the level of CYP1A2 mRNA is increased 3×. The physician lowers the dose to 10 mg per day and dosing to once every other day. Two weeks later the level of CYPIA1 mRNA is increased 15× and the level of CYP1A2 mRNA is increased 6× compared to baseline levels. Based on the continued increase in both biomarkers despite a dose and dosing reduction, the physician immediately instructs the patient to stop therapy.

### Example 22

### Monitoring Plasma Biomarkers Related to Development of PAH

During the course of 3-months of treatment of atopic dermatitis with oral laquinimod, a patient comes in for a routine follow up and complains of shortness of breath. The patient is responding well to therapy and has had no other adverse events. The doctor orders a blood test for serotonin. There is 950 ng/ml serotonin in the blood (normal range is 101-283 ng/ml). Given the findings, the doctor is concerned that systemic CYP1A1 signaling is too high and immediately discontinues therapy and recommends routine echocardiograms to evaluate the possible development of PAH.

### Example 23

### Administering an AhR Antagonist in Combination With an AhR Agonist

During the course of therapy with a nanoparticulate form of synthetic indirubin for the treatment of gastritis, a patient complains of ad adverse event, in this case shortness of breath and difficulty doing physical exercise. The patient is responding well to therapy but the doctor is concerned about possible development of additional PAH-like symptoms. The doctor maintains the indirubin therapy but prescribes the patient an inhalable AhR antagonist, in this case inhaled 1-Methyl-N-[2-methyl-4-[2-(2-methylphenyl)diazenyl]phenyl-1H-pyrazole-5-carboxamide (CH223191) that is designed to reduce AhR signaling in the lung epithelium. After two weeks of combination therapy, the patient no longer has shortness of breath can exercise without issue and is still responding well to therapy and both drugs are maintained.

### Example 24

Label Warning Related to the Risk Of Hypoxic Conditions in Combination with AhR Agonists

Prior to beginning therapy of a FICZ derivative eye-drop for the treatment of severe unresponsive uveitis a patient comes into the doctor's office. The patient is a candidate for therapy based on the label of the drug but lives in Denver and is an avid mountain climber with sleep apnea so is routinely exposed to hypoxic conditions. The label of the drug warns about possible development of PAH like symptoms if the product is used in patients who frequently experience hypoxia and so the doctor prescribes the drug but requires that the patient return to the office every month for echocardiograms to monitor for development of PAH symptoms.

### Example 25

Cardiac Risk Mitigation Program During AhR Agonist Therapy

Based on the label of an orally administered tablet containing linomide, a patient must enroll in a risk evaluation and mitigation strategy (REMS). In the case of this drug, patients must comply with ongoing echocardiogram monitoring requirements. Although the patient was not deemed high risk of development of a cardiac adverse event at baseline, after 6-months the data from the echocardiogram show signs of valvar heart disease as indicated by moderate aortic regurgitation and restrictive valve motion. The prescribing physician evaluates the echocardiogram an decides the patient should be discontinued. The physician passes this information along to the REMS program by logging the data into a secure server. Subsequently the server alerts the patient and the prescribing pharmacy that the patient is no longer eligible to refill their linomide tablet prescription.

1. A method for treatment with an aryl hydrocarbon receptor (AhR) agonist compound, comprising:
prior to treating a subject with an AhR agonist compound, providing or instructing to provide a sample from a subject;
testing or instructing to test the sample for a diagnostic marker related to aryl hydrocarbon receptor biology; and
based on a result from the testing or the instructing to test, performing one or more of (i) administering the AhR agonist compound, (ii) not administering the AhR agonist compound, and (iii) altering a dose or a dosing protocol of the AhR agonist compound.

2. The method of claim 1, wherein the testing or instructing to test is for a diagnostic marker selected from the group consisting of:
a. an AhR agonist compound in a sample of skin, stool, urine, or plasma obtained from the subject;
b. tryptophan in a sample of skin, stool, urine or plasma obtained from the subject;
c. a cytochrome P450 (CYP) enzyme selected from CYP1A1, CYP1A2, CYP1B1, and CYP19A1 in a colon sample or biopsy, an intestinal biopsy, a skin biopsy or peripheral blood mononuclear cells (PBMCs) obtained from the subject;
d. an aryl hydrocarbon receptor repressor (AhRR) mRNA or protein in a colon sample or biopsy, an intestinal biopsy, a skin biopsy or peripheral blood mononuclear cells (PBMCs) obtained from the subject;
e. nuclear localizing AhR in a colon sample or biopsy, an intestinal biopsy or a skin biopsy obtained from the subject;
f. a level of IL-22 mRNA or protein in a sample of plasma, a colon sample of biopsy, an intestinal biopsy or a skin biopsy obtained from the subject; and
g. a polymorphism in caspase recruitment domain 9 (CARD9) in a sample taken from the subject.

3. The method of claim 1, whereby the method achieves an improvement in response to treatment with the AhR agonist compound that is at least about 25% increased relative to a subject treated with the same AhR agonist compound that did not receive a test for the diagnostic marker.

4. A method for preventing or mitigating an adverse event prior to or during treatment with a compound with activity as an aryl hydrocarbon receptor (AhR) agonist, comprising:
prior to or while treating a subject with an AhR agonist compound, providing or instructing to provide a sample from a subject;
testing or instructing to test the sample for a diagnostic marker related to aryl hydrocarbon receptor biology; and
based on a result from the testing or the instructing to test, performing one or more of
i. administering the AhR agonist compound according to a schedule selected to reduce likelihood or severity of an adverse event;
ii. administering the AhR agonist compound at a dose selected to reduce likelihood or severity of an adverse event;
iii. not administering the AhR agonist compound;
iv. administering a treatment other than an AhR agonist; and
v. administering an AhR antagonist or a compound that induces CYP.

5. The method of 4, wherein the testing or instructing to test is for a diagnostic marker selected from the group consisting of:
a. a polymorphism in AhR, ARNT, HIF-1 α or HIF-2α, AhRR, BMPR2, CYP1A1, CYP1A2, CYP1B1 and CYP19A1 or a promoter thereof;
b. ability for microsomes or cells from the subject to metabolize an AhR agonist in vitro or in vivo; and
c. ability of an AhR agonist to induce AhR translocation to the nucleus or to induce CYP1A1, CYP1A2, CYP19A1 and/or CYP1B1 expression in vitro or in vivo.

6. The method of claim 4, wherein said performing comprises (i), (ii) or (v), and said administering according to (i), (ii) or (v) reduces occurrence or severity of an adverse event in a subject compared to subjects that are not administered the AhR agonist compound or a compound that induces a CYP enzyme.

7. The method of claim 6, wherein said administering according to (i), (ii) or (v) reduces occurrence or severity of an adverse event in a subject by at least about 20%.

8. A method for preventing or mitigating an adverse event during treatment with a compound with activity as an aryl hydrocarbon receptor (AhR) agonist, comprising:
during treatment of a subject with an AhR agonist compound, providing or instructing to provide a sample from a subject;
testing or instructing to test the sample for a diagnostic marker related to aryl hydrocarbon receptor biology; and
based on a result from the testing or the instructing to test, performing one or more of
i. administering the AhR agonist at a dosing frequency that is reduced relative to a clinically approved dosing frequency or a previously planned dosing frequency;
ii. administering the AhR agonist at a dose that is reduced relative to a clinically approved dose or a previously planned dose;
iii. administering an initial starting dose of the AhR agonist followed by a subsequent dose that is greater than the initial starting dose;
iv. discontinuing use of the AhR agonist;
v. continuing use of the AhR agonist in conjunction with safety monitoring;
vi. administering a treatment other than an AhR agonist;
vii. co-administration of an AhR antagonist or of a compound that induces CYP1A, CYP1A2, CYP1B1 or CYP19A1;
viii. co-administration of a concomitant medication;
ix. discontinuing of a concomitant medication, food, drug or activity; and
x. no change in treatment.

9. The method of claim 4, wherein the testing or instructing to test is for a diagnostic marker selected from the group consisting of:
a. plasma, urine, colon or skin biopsy level of the administered AhR agonist or a metabolite thereof;
b. a plasma liver enzyme, such as alanine aminotransferase (ALT) or aspartate aminotransferase (AST);
c. PBMC expression of nuclear AhR or increased AhRR, CYP1A1 CYP1A2, CYP1B1 or CYP19A1 and/or CYP1B1 expression relative to a healthy subject;
d. systolic blood pressure, mean pulmonary arterial pressure (mPAP), pulmonary arterial systolic pressure (PASP), left atrial dimension (LAD), left ventricle end-diastolic diameter (LVEDD), left ventricular ejection fraction (LVEF), inferior vena cava dimension, right atrial area (RA area), tricuspid annular plan systolic excursion (TAPSE), tricuspid regurgitation pressure gradient (TRPG), aortic regurgitation, mitral regurgitation, valve thickening, restrictive valve motion, shortness of breath and/or fatigue;
e. hematocrit or plasma erythropoietin (EPO) level; and
f. plasma or urine level of serotonin, melatonin, 17B-estradiol (E2) or 16α-hydroxyestrone.

10. The method of claim 8, wherein said performing comprises (i), (ii), (iii), (v), (vii), (viii), or (ix), and said administering according to (i), (ii) or (v), said continuing according to (v), said co-administration according to (vii) or (viii) or said discounting according to (ix) reduces occurrence or severity of an adverse event in a subject compared to subjects that are not administered the AhR agonist compound, not administered a compound that induces a CYP enzyme or a concomitant medication, and/or continuing a concomitant medication, food, drug or activity.

11. The method of claim 10, wherein said reduces occurrence or severity of an adverse event in a subject is by at least about 20%.

12. The method of clam 8, wherein said continuing use of the AhR agonist in conjunction with safety monitoring comprising continuing use with a safety monitoring program selected from an echocardiogram, a walk test for a defined period of time and a questionnaire regarding shortness of breath and/or ability to exercise without fatigue.

13. A method for preventing or mitigating an adverse event prior to or during treatment with a compound with activity as an aryl hydrocarbon receptor (AhR) agonist, comprising:
consulting a product label that prohibits certain concomitant medications, drugs, foods, diets, supplements and/or activities that may increase AhR signaling, increase AhR agonist absorption or interfere with the ability to metabolize an AhR agonist, and based on said consulting, determining whether to administer, to not administer or to administer with an adjusted a dose range or frequency of the AhR agonist to a subject based on likelihood of an adverse event for the subject.

14. A method for preventing or mitigating an adverse event prior to or during treatment with a compound with activity as an aryl hydrocarbon receptor (AhR) agonist, comprising:
consulting a product label that contraindicates the use of the AhR agonist in patients with known medical conditions, predispositions or family history that may increase the likelihood of developing a cardiovascular disease, including pulmonary arterial hypertension and valvular heart disease.

15. A method for reducing systemic exposure or Cₘₐₓ of an AhR agonist, comprising:
administering a compound that induces a CYP enzyme capable of metabolizing a to-be-administered AhR agonist, wherein said administering is prior to administration of an oral dosage form containing an AhR agonist.

16. The method of claim 15, wherein the compound that induces a CYP is an AhR agonist.

17. The method of claim 16, where the AhR agonist that induces a CYP is the same as the AhR agonist in the oral dosage form, wherein the AhR agonist that induces a CYP is administered at a dose lower than a dose of the AhR agonist in the oral dosage form.

18. The method of claim 15, whereby said administering reduces occurrence or severity of an adverse event in a subject compared to subjects that are not administered a compound that induces a CYP enzyme.

19. The method of claim 18, wherein said administering reduces occurrence or severity of an adverse event in a subject by at least about 20%.

20. The method of claim 4, wherein the adverse event is selected from the group consisting of folliculitis, essential hypertension, pulmonary hypertension, pulmonary arterial hypertension, pneumonitis, respiratory failure, myocardial ischemia, myocardial infarction, pericarditis, pleuritis, peritonitis, pneumonitis, chest pain, abdominal pain, diarrhea, vomiting, intussusception, joint pain, back pain, liver damage, changes in liver enzymes, infection, peripheral neuropathy, headache, pancytopenia, and leukopenia.

21. A method of treating one or more patients with an AhR agonist, comprising:
providing a data storage facility comprising a database of patient records, each patient record having a medication authorization field for entering a first prescription for the AhR agonist to treat the patient;
a central controller having one or more processors coupled to a communication network, which central controller is coupled to the data storage facility to read and write data to the data storage facility via the network; and
wherein the central controller controls transmission and receipt of data to and from the data storage facility via the network,
the central controller being programed to output via the network a first authorization of a first prescription of AhR agonist to a patient previously subjected to one or more initial medical tests, each providing an initial medical test result,
wherein the initial medical test is selected from the group consisting of a medical examination by a physician, a genetic test, a physiological function test, and a medical imaging test,
wherein output of the first authorization is dependent upon satisfactory results of one or more of the initial medical tests entered into each patient's record, and
further programed to schedule one or more subsequent tests for each patient prior to allowing entry of a prescription in the medication authorization field,
wherein at least one of said subsequent medical tests is an echocardiographic imaging test which echocardiographic imaging test is performed in a manner which provides measurements of dimensions of one or more internal heart structures and heart flow-rate, and the patient receives or continues to receive medication only on entry of satisfactory echocardiography assessment results,
wherein the central controller inhibits the authorization output of the first or subsequent prescriptions upon the entry of unsatisfactory test results;
wherein the central controller manages one or more aspects of the authorized prescription for the patient selected from the group consisting of dosage amount; dosing regimen; and intended time period of use, whereby overuse or misuse of the AhR agonist is inhibited and wherein aggregated and analyzed data is reported to a regulatory agency.
